(19) United States
(12) Patent Application Publication
(10) Pub. No.: US 2017/0319669 A1
(43) Pub. Date: Nov. 9, 2017

(54) METHOD OF ENHANCING VIRAL-MEDIATED GENE DELIVERY

(71) Applicant: Wayne State University, Detroit, MI (US)

(72) Inventors: Zhuo-Hua PAN, Troy, MI (US); Shengjie CUI, Detroit, MI (US); Gary ABRAMS, Detroit, MI (US)

(21) Appl. No.: 15/586,164

(22) Filed: May 3, 2017

Related U.S. Application Data
(60) Provisional application No. 62/331,281, filed on May 3, 2016.

(51) Int. Cl. A61K 38/57; A61K 38/17; A01K 67/027; A61K 38/18; A61K 38/19; A61K 48/00

(52) U.S. Cl. CPC A61K 38/57; A61K 38/185; A61K 38/179; A61K 38/19; A01K 67/0275; A61K 38/18; A61K 48/00; C12N 2799/021

(57) ABSTRACT

The invention provides methods for enhancing the delivery of viral vectors to the eye of a subject by administering a proteasome inhibitor or and a viral vector ending a gene of interest to the eye.

### RELATED APPLICATIONS

This application claims priority to, and the benefit of U.S. Provisional Application No. 62/331,281 filed on May 3, 2016, the contents of which is incorporated by reference in its entirety.

### GOVERNMENT SUPPORT

This invention was made with government support under EY017130 awarded by National Institutes of Health. The government has certain rights in the invention.

### INCORPORATION BY REFERENCE OF SEQUENCE LISTING

The contents of the text file name “RTRO-706-001US_ST25” which was created on May 3, 2017 and is 56 KB in size, are hereby incorporated by reference in their entirety.

### FIELD OF THE INVENTION

This invention relates generally to methods for improving the efficacy of gene delivery such as viral transduction of cells. More particularly, the present invention provides methods and materials useful for safely and reliably improving the efficiency of methods for transducing cells, such as retina cells, with viruses and/or viral vectors.

### BACKGROUND OF THE INVENTION

The eye is a complex optical system that detects light, converts the light to a set of electrical signals, and transmits these signals to the brain, ultimately generating a representation of our world. Ocular diseases and disorders can cause diminished visual acuity, diminished light sensitivity, and blindness.

Low transduction efficiency is a major challenge for viral mediated gene therapy in retinal neurons, Thus, there exists a long-felt need for methods to enhance the delivery of viral vectors to the eye

### BRIEF DESCRIPTION OF THE DRAWINGS

FIG. 1A-B is a series of photographs and a graph depicting the effects of proteasomes on the AAV-mediated expression of transgene (mCherry) in retinal bipolar cells one month after virus injection. FIG. 1A: Representative images of virus transduced retinal bipolar cells. Targeted expression of mCherry in retinal bipolar cells was achieved by rAAV2 vectors carrying a mGluR6 promotor. Virus vectors (1 μl) at the titer of 5×10¹² vg (viral-genome contacting particle)/ml with or without containing proteasome inhibitors were intravitreally injected into the eyes of C57BL/6J mice at about one month of age. Animals were euthanized one month after virus injection for assessing the expression of mCherry. DOX: doxorubicin; Acla: Aclarubicin; MG: MG132. FIG. 1B. Statistical data for evaluating the fluorescence intensity of mCherry in bipolar cells one month after virus injection. The expression of mCherry in bipolar cells were significantly increased with the co-injection of DOX at concentrations≧300 μM.

FIG. 2A-B is a series of photographs and a graph depicting the effects of DOX on the AAV-mediated expression of transgene (mCherry) in retinal bipolar cells three months after virus injection. FIG. 2A. Representative images of virus transduced retinal bipolar cells three months after virus injection. Virus vectors were co-injected with DOX at different concentrations. FIG. 2B. Statistical data for evaluating the fluorescence intensity of mCherry in bipolar cells three months after virus injection. The expression of mCherry in bipolar cells were significantly increased with the co-injection of DOX at concentrations≧200 μM.

FIG. 3A-B is a series of photographs and a pair of graphs, depicting the effects of DOX on the morphological properties of virus transduced retina. FIG. 3A. Representative images of retinal vertical sections three months after the co-injection of virus with different concentration of DOX. At high DOX concentrations, bipolar cell layer appears thinner. Red: bipolar cells transduced with mCherry. Green: PKC antibody labeled rod bipolar cells. Blue: DAPI stained nuclei. FIG. 3B. Statistical data for the comparison of the thickness of photoreceptor cell body layer and bipolar cell layer. Animals were euthanized three months after the virus injection. With the co-injection of DOX at the concentration of 300 or 500 μM, bipolar cell layer was statistically thinner than that of control.

FIG. 4A-B is a series of photographs and a graph, depicting the effects of DOX on retinal ganglion cells. FIG. 4A. Representative images for evaluating the density of retinal ganglion cells with DAPI staining after virus transduction in bipolar cells with and without DOX at different concentrations. Animals were euthanized one and three months after virus injection. FIG. 4B. Statistical data for evaluating the density of retinal ganglion cells one and three months after virus injection with and without doxorubicin (DOX). Three months after virus injection at 300 μM and 500 μM DOX, retinal ganglion cell density was statistically lower than that of control.

### SUMMARY OF THE INVENTION

The invention provides a solution for the long-felt need for methods to enhance or improve therapeutic gene delivery to the eye.

The present invention features a method of enhancing the delivery of a gene of interest to an eye of a subject by administering a proteasome inhibitor and a viral vector encoding a gene of interest to the eye.

The proteasome inhibitor is doxorubicin, aclarubicin, bortezomib, lactacystin, disulfiram epigallocatechin-3-gallate marizomib (salinosporamide A), oprozomib (ONX-0912), delanzomib (CEP-18770) epoxomicin, MG132, beta-hydroxy beta-methylbutyrate or carfilzomib.

Preferably, the proteasome inhibitor is a doxorubicin, aclarubicin or MG132.

The gene of interest is an opsin. Examples of opsin genes include, but are not limited to, channelrhodopsins (i.e., channelrhodopsin-1, channelrhodopsin-2, Volvox carteri channelrhodopsins 1 or 2), melanopsin, pineal opsin, photopsins, halorhodopsin, bacteriorhodopsin, proteorhodopsin, or any functional variants or fragments thereof.

The opsin is channelrodopsin, halorhopdopsin or a functional variant or fragments therefore.

The viral vector is a AAV viral vector (i.e., recombinant AAV or rAAV) that encodes a gene of interest (i.e., transgene).

For example, the AAV viral vector is AAV2, AAV3, or AAV8. In some embodiments of the method of the disclosure, the viral vector is AAV2.

Preferably, the gene of interest is operably linked to a cell-specific promoter. For example, the cell-specific promoter is mGluR6, NK-3, and Pcp2 (L7). In some embodiments, the cell specific promoter is mGluR6.

The viral vector may be encapsulated in a nanoparticle, a polymer, or a liposome.

In one aspect, the proteasome inhibitor and the viral vector are delivered concurrently or sequentially.

The present invention provides a method in which the viral vector is delivered to a retinal cell. The retinal cell is a retinal ganglion cell, a retinal horizontal cell, a retinal bipolar cell, an amacrine cell, a photoreceptor cell, a Miller glial cell, or a retinal pigment epithelial cell.

In one aspect, the proteasome inhibitor and the viral vector is administered to the vitreous of the eye.

In other aspects, the proteasome inhibitor and the viral vector are administered by a route wherein the administration is by injection or infusion.

In a further aspect, the proteasome inhibitor and the viral vector are administered by a route that is not subretinal.

The present invention further provides a method of increasing or restoring light sensitivity in a subject comprising administering the proteasome inhibitor and the viral vector that encodes an opsin to the vitreous of the eye. The present invention also provides a method of improving or restoring vision in a subject comprising administering a proteasome inhibitor and the viral vector that encodes an opsin to the vitreous of the

Uses of a composition comprising a proteasome inhibitor for treating an ocular disease or disorder in a subject are also provided herein.

The subject is suffering from an ocular disease or disorder. The ocular disease is retinoblastoma, ocular melanoma, diabetic retinopathy, hypertensive retinopathy, any inflammation of the ocular tissues. Preferably, the ocular disease or disorder is associated with photoreceptor degeneration.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control.

In addition, the materials, methods, and examples described herein are illustrative only and are not intended to be limiting.

Other features and advantages of the invention will be apparent from and are encompassed by the following detailed description and claims.

### DETAILED DESCRIPTION

The present invention generally relates to improved gene therapy compositions and methods of using the same to treat, prevent, or ameliorate disease. One significant challenge for gene therapy is to increase the transduction efficiency of cells comprising a therapeutic gene that will be delivered to a subject.

The present invention is based, in part, on the unexpected discovery that proteasome inhibitors were found to enhance viral mediated transduction efficiency. Accordingly, the present invention addresses an unmet clinical need for improving the efficiency of gene therapy in the treatment of diseases.

The present invention provides methods for enhancing the efficiency of viral mediated gene delivery by administering a proteasome inhibitor and a therapeutic agent. The therapeutic agent is a viral vector encoding a gene of interest. Preferably, proteasome inhibitor and a therapeutic agent is delivered to the eye.

In some embodiments, the proteasome inhibitor and the therapeutic agent may be delivered to the vitreous for enhanced delivery to the retina and retinal cells. The retinal cells include, for example, photoreceptor cells (e.g., rods, cones, and photosensitive retinal ganglion cells), horizontal cells, retinal bipolar cells, amacrine cells, retinal ganglion cells, Müller glial cells, and retinal pigment epithelial cells. In other embodiments, the proteasome inhibitor and the therapeutic agent may be delivered to, for example, the posterior segment, the anterior segment, the sclera, the choroid, the conjunctiva, the iris, the lens, or the cornea.

The retina is a complex tissue in the back of the eye that contains specialized photoreceptor cells called rods and cones. The photoreceptors connect to a network of nerve cells for the local processing of visual information. This information is sent to the brain for decoding into a visual image. The retina is susceptible to a variety of diseases, including macular degeneration, age-related macular degeneration (AMD), diabetic retinopathy (DR), retinitis pigmentosa (RP), glaucoma, and other inherited retinal degenerations, uveitis, retinal detachment, and eye cancers (ocular melanoma and retinoblastoma). Each of these can lead to visual loss or complete blindness.

Delivery of therapeutic compounds to the retina is a challenge, due to the complex structure of the eye. Intravitreal injection and vitreal delivery devices are frequently used to deliver therapeutic compounds to the retina, however the efficiency of delivery is impaired by the inner limiting membrane (ILM) and the multiple layers of cells of the retina.

The proteasome inhibitor and the therapeutic agent may be delivered to the eye by any method known in the art. Routes of administration include, but are not limited to, intravitreal, intracameral, subconjunctival, subtenon, retrobulbar, posterior juxtascleral, or topical. Delivery methods include, for example, injection by a syringe and a drug delivery device, such as an implanted vitreal delivery device (i.e., VITRASERT®).

Preferably, the proteasome inhibitor and the therapeutic agent is administered to the vitreous by intravitreal injection for delivery to the retina.

In one embodiment, the proteasome inhibitor is administered concurrently or sequentially with the therapeutic agent. For concurrent administration, the proteasome inhibitor can be formulated with the therapeutic agent in a single composition suitable for delivery, for example, injection, by methods known in the art. Alternatively, the proteasome inhibitor can be injected in separate compositions, simultaneously or sequentially. In a preferred embodiment, the proteasome inhibitor may be administered prior to administration of the therapeutic agent.

Such formulations comprise a pharmaceutically and/or physiologically acceptable vehicle, diluent, carrier or excipient, such as buffered saline or other buffers, e.g., HEPES, to maintain physiologic pH. For a discussion of such components and their formulation, see, generally, Gennaro, A E., Remington: The Science and Practice of Pharmacy, Lippincott Williams & Wilkins Publishers; 2003 or latest edition). See also, WO00/15822. If the preparation is to be stored for long periods, it may be frozen, for example, in the presence of glycerol.

The dosage of a proteasome inhibitor thereof to be administered can be optimized by one of ordinary skill in the art. Delivery to certain target ocular tissues may require lower doses of a proteasome inhibitor or higher doses of a proteasome inhibitor, depending on the location of the target tissue, intervening ocular structures, and ability of the agent to penetrate the target tissue. Preferably, the dose of the proteasome inhibitor administered is about 50 to 2000 μM per eye, preferably 100 to 1000 μM. More preferably 200 to 800 μM per eye. For example, 50, 100, 150, 200, 250, 300, 350, 400, 450, 500, 550, 600, 650, 700, 750, 800, 850, 900, 950 or 1000 μM of a proteasome inhibitor is delivered to an eye

Proteasome inhibitors are known in the art. For example, a proteasome inhibitor is doxorubicin, aclarubicin, bortezomib, lactacystin, disulfiram epigallocatechin-3-gallate marizomib (salinosporamide A), oprozomib (ONX-0912), delanzomib (CEP-18770) epoxomicin, MG132, beta-hydroxy beta-methylbutyrate or carfilzomib. In preferred embodiments, the proteasome inhibitor is doxorubicin, aclarubicin or MG132.

In some embodiments, the methods for enhanced delivery disclosed herein may provide increased efficacy of a therapeutic agent. Increased efficacy of the therapeutic agent can be determined by measuring the therapeutic effect of the therapeutic agent. Treatment is efficacious if the treatment leads to clinical benefit such as, alleviation of a symptom in the subject. For example, in a degenerative retinal disease, such as retinitis pigmentosa, treatment is efficacious when light sensitivity or another aspect of vision is improved or restored. When treatment is applied prophylactically, “efficacious” means that the treatment retards or prevents an ocular disease or disorder or prevents or alleviates a symptom of clinical symptom of an ocular disease or disorder. Efficaciousness is determined in association with any known method for diagnosing or treating the particular ocular disease or disorder.

The gene of interest to be delivered by the methods described herein are any gene of interest (i.e., therapeutic transgene) known in the art for treating, alleviating, reducing, or preventing a disease. Preferably, the gene of interest (i.e., therapeutic transgene) is known in the art for treating, alleviating, reducing, or preventing a symptom of an ocular disease, an ocular disorder, or an ocular condition.

Examples of nucleic acids suitable for use in the methods described herein include, but are not limited to, viral vectors encoding therapeutic transgenes (i.e., channelopsins, or halorhodopsin), RNA interference molecules (i.e., short hairpins, siRNA, or microRNAs). In a particularly preferred embodiment, the therapeutic agents are viral vectors encoding transgenes for gene therapy. Particularly preferred viral vectors are rAAV vectors that encodes a rhodopsin such as channelopsins or halorhodopsins for expression in the retina to restore light sensitivity.

Examples of antibodies suitable for use in the methods described herein include, but are not limited to, ranibizumab (Lucentis®), VEGF antibodies (Eylea®), bevacizumab (Avastin®), infliximab, etanercept, and adalimumab.

Any of the agents described herein may be optionally encapsulated in a carrier, such as a nanoparticle, a polymer, or a liposome. These carrier agents may serve to further enhance the delivery of the therapeutic agent to the eye. In some aspects, the carrier agents may alter the properties of the therapeutic agents, such as increasing the stability (half-life) or providing sustained-release properties to the therapeutic agents. Alternatively, the carrier may protect the therapeutic agent from the proteolytic activities of plasmin if formulated in the same composition for delivery.

As a large number of ocular diseases and disorders result from aberrant gene expression in various ocular tissues, gene therapy possesses increasing potential as an effective therapy. However, the efficacy of gene therapy in the eye has been limited due to the challenges of effective delivery and transduction of the therapeutic viral vectors throughout any ocular tissue.

Thus, the present invention provides methods for increased efficiency of delivery of transgenes to the eye for treating an ocular disease or disorder, or for restoring or improving vision. Transgenes of particular interest for restoration of photosensitivity or vision include photosensitive proteins, such as opsin genes or rhodopsin genes. As used herein, “transgene” refers to a polynucleotide encoding a polypeptide of interest, wherein the polynucleotide is present in a nucleic acid expression vector suitable for gene therapy (e.g., a viral vector such as AAV).

Previous studies have shown that injection of a recombinant adeno-associated viral vector encoding a transgene, such as channelopsin-2, results in poor delivery of the vector and low expression of Chop2 in the inner retinal cells, especially bipolar cells. In non-human primates, AAV-mediated gene transfection was found to be more efficient in peripheral retina, fovea, and along blood vessels, suggesting that inner limiting membrane (ILM), which is the boundary between the retina and the vitreous space, is a major barrier (Ivanova et al., 2010).

The present invention provides a solution to this problem by using a proteasome inhibitor to inhibit or reduce proteasome dependent virus degradation. Accordingly, therapeutic agents will have greater accessibility to the retina, specifically the cells of the inner retina such as the retinal bipolar cells, retinal ganglion cells, Miller glial cells, and retinal pigment epithelial cells. The methods described herein provide enhanced delivery of therapeutic viral vectors. The enhanced delivery of viral vectors is demonstrated by increased transduction efficiency, increased expression of the therapeutic transgene (i.e., Chop2), and increased efficacy of the therapeutic compound (i.e., increased light sensitivity or restoration of vision).

Nucleic acid expression vectors suitable for use in gene therapy are known in the art. For example, the nucleic acid expression vector is a viral vector. The viral vectors can be retroviral vectors, adenoviral vectors, adeno-associated vectors (AAV), or lentiviral vectors, or any engineered or recombinant viral vector known in the art. Particularly preferred viral vectors are adeno-associated vectors, for example, AAV-1, AAV-2, AAV-3, AAV-4, AAV-5, AAV-6, AAV-7, AAV-8, AAV-9, AAV-10, AAV-11, AAV-12 or any engineered or recombinant AAV known in the art. In a particularly preferred embodiment, the vector is recombinant AAV-2 (rAAV2).

In some embodiments, a recombinant adeno-associated viral (rAAV) vector comprises a capsid protein with a mutated tyrosine residue which enables to the vector to have improved transduction efficiency of a target cell, e.g., a retinal bipolar cell (e.g. ON or OFF retinal bipolar cells; rod and cone bipolar cells). In some cases, the rAAV further comprises a promoter (e.g., mGluR6, or fragment thereof) capable of driving the expression of a protein of interest in the target cell.

In one embodiment, a mutation may be made in any one or more of tyrosine residues of the capsid protein of AAV 1-12 or hybrid AAVs. In specific embodiments, these are surface exposed tyrosine residues. In a related embodiment the tyrosine residues are part of the VP1, VP2, or VP3 capsid protein. In exemplary embodiments, the mutation may be made at one or more of the following amino acid residues of an AAV-VP3 capsid protein: Tyr252, Tyr272, Tyr444, Tyr500, Tyr700, Tyr704, Tyr730; Tyr275, Tyr281, Tyr508, Tyr576, Tyr612, Tyr673 or Tyr720. Exemplary mutations are tyrosine-to-phenylalanine mutations including, but not limited to, Y252F, Y272F, Y444F, Y500F, Y700F Y704F, Y730F, Y275F, Y281F, Y508F, Y576F, Y612G, Y673F and Y720F. In a specific embodiment, these mutations are made in the AAV2 serotype. In some cases, an AAV2 serotype comprises a Y444F mutation and/or an AAV8 serotype comprises a Y733F mutation, wherein 444 and 733 indicate the location of a point tyrosine mutation of the viral capsid. In further embodiments, such mutated AAV2 and AAV8 serotypes encode a light-sensitive protein and also comprise a modified mGluR6 promoter to drive expression of such light-sensitive protein. Such AAV vectors are described in, for example Petrs-Silva et al., Mol Ther., 2011 19:293-301).

In some embodiments, the expression of the therapeutic transgene is driven by a constitutive promoter, i.e., CAG promoter, CMV promoter, LTR. In other embodiments, the promoter is an inducible or a cell-specific promoter. Cell type-specific promoters that enable transgene expression in specific subpopulations of cells, i.e., retinal neuron cells or degenerating cells, may be preferred. These cells may include, but are not limited to, a retinal ganglion cell, a photoreceptor cell, a bipolar cell, a rod bipolar cell, an ON-type cone bipolar cell, a retinal ganglion cell, a photosensitive retinal ganglion cell, a horizontal cell, an amacrine cell, an AII amacrine cell, or a retinal pigment epithelial cell. Cell type-specific promoters are well known in the art. Particularly preferred cell type-specific promoters include, but are not limited to mGluR6, NK-3, and Pcp2(L7). Cell type-specific promoters modified using recombinant DNA techniques known in the art to increase efficiency of expression and selective targeting are also encompassed in the present invention. For example, a modified mGluR6 promoter contains a combination of regulatory elements from the mGluR6 gene, as described in U.S. Publication NoUS 2017-0021038 A1, hereby incorporated by reference in its entirety.

In one embodiment of the present invention, the gene of interest (i.e, therapeutic transgene) can be any light-sensitive opsin. The opsin family of genes includes vertebrate (animal) and invertebrate opsins. Animal opsins are G-protein coupled receptors (GPCRs) with 7-transmembrane helices which regulate the activity of ion channels. Invetertebrate rhodopsins are usually not GPCRs but are light-sensitive or light-activated ion pumps or ion channels. Modified mGluR6 gene promoter

(SEQ ID NO: 2)
caagcaggaggctgctgtgtgctgggagctgtcaggctcgtcctgaacag
ggaagggcccatccacctcccaaacccagtttatgcagtccttcgcaatg
tcaggctcagggcctggcaccagccaagctccccacccttcccactgtta
aaatggataggagcagggctaggcccagcctgttgactctgggcttccac
caggagaagtggttctggcagtagaaactatcggggcctgggagaggcgg
gggaagagagaaaggtggcatgtttcttgcttgctccctctaccagcctt
gtccaaatccccgcagccaccctaatccagcctgtctaatggagcccaag
ccggctcaggccctcggacgaggagcctgctaatccctgtggctaggagc
tcaccacctgtctccaggacgccctttgctctcttggcatcagagagcca
aatcctgggcctcggatggggggatgataaaagcatcttttggccaagcc
ccctcaccttggcctccacgatgagatggggagttaggtgcagagagcgt
tggcacagtgagcaccgcagctcgagtggctgcctcagacccagagcccg
aggagactttatacggagccagaacgaccccgcggggttccatcctccca
agcaataggcgggagtgggagctgcgaggaaagccggcccctcccctccc
tccatccaaggcagtgtgggctgtttgtttcatgccattctgggtgtgaa
tcctgatgcccacacatgccagctgcatgcacttgggcaactcaactcac
tcctcgagggctgtttctcgactgcagggtgttgtaagttcgctaatact
aaaggcttctccctcctggccccttcctgcccctcgctcttcctcctctt
ccttaggccctcccagctcaggcagcccctgccccctgcagggttctgca
aggagaaagctggggaataccttaggcaactgcagtcaggagcactggtg
gccaggacagagacagagagacagaaaaggggtcagggacagagagagat
aaccgcagggagagacaggaagggacagagacagaaaagataccaagaag
aggacagaggcagaaagccagggacagagactgagaaacagagacctaga
ggcagaagaagactgagatagagatggacagagattgtgtcagacacagc
cccagagacagccagacagtctgagtcagacgcaaaccaaagacaagaaa
acaggaaaacagacccagagattgggagagggaggggaaggagatgcggg
gagagccagcaccgccaccccccacactcaggaggggtctccaccctcgg
agcggtctctcatccctccctagaatccttaaatcctctctcgctcaggg
cctcggccgcatctgtcacagacttgtcctgaaccgacagcggctggcgc
aggtgactggcttggggcgggagcctgggtgtgcgctggggatggacccc
gaggaagaggggccaagctgtcgggaagcggcagggctggaggggtggag
gcagtggtcgggcgggaccccgggcgacagggttcggcgcttgtaagagc
gagacggaggcccgggcaggccggctgagctaactccccagagccgaagt
ggaaggcgcgccccgagcgccttctccccaggaccccggtgtccctcccc
gcgccccgagcccgcgctctccttcccccgccctcagagcgctccccgcc
cctctgtctccccgcagcccgctagacgagccg.

As referred to herein, an opsin gene or light-sensitive protein includes, but is not limited to, channelrhodopsins, or channelopsins, (i.e., ChR1, ChR2, vChR1 from Volvox carteri, vChR2, and other variants identified from any vertebrate, invertebrate, or microbe), halorhodopsins (NpHR), melanopsins, pineal opsins, photopsins, bacteriorhodopsins, proteorhodopsins and functional variants or chimeras thereof. A light-sensitive protein of this invention can occur naturally in plant, animal, archaebacterial, algal, or bacterial cells, or can alternatively be created through laboratory techniques. Examples of opsin genes are discussed in further detail below.

Examples of channelrhodopsins, or channelopsins, as transgenes in the present invention include channelrhodopsins Chop1 (also known as ChR1) (GenBank accession number AB058890 (SEQ ID NO: 3)/AF385748 (SEQ ID NO: 4)) and Chop2 (also known as ChR2) (GenBank accession number AB058891 (SEQ ID NO: 5)/AF461397 (SEQ ID NO: 6)) are two rhodopsins from the green alga Chlamydomonas reinhardtii (Nagel, 2002; Nagel, 2003).

A nucleic acid sequence encoding an exemplary Chop1 of the disclosure comprises or consists of GenBank accession number AB058890:

| (SEQ ID NO: 3) | |
|---|---|
| 1 | cttgactacg cttcgctgta ataatagcag cgccacaagt agtgtcgcca gacaactctc |
| 61 | actttgagct tgagcacacc gctgagcccc gatgtcgcgg aggccatggc ttcttgccct |
| 121 | agcgctggca gtggcgctgg cggccggcag cgcaggagcc tcgactggca gtgacgcgac |
| 181 | ggtgccggtc gcgactcagg atggccccga ctacgttttc caccgtgccc acgagcgcat |
| 241 | gctcttccaa acctcataca ctcttgagaa caatggttct gttatttgca tcccgaacaa |
| 301 | cggccagtgc ttctgcttgg cttggcttaa atccaacgga acaaatgccg agaagttggc |
| 361 | tgccaacatt ctgcagtgga ttacttttgc gctttcagcg ctctgcctga tgttctacgg |
| 421 | ctaccagacc tggaagtcta cttgcggctg ggaggagatt tacgtggcca cgatcgagat |
| 481 | gatcaagttc atcatcgagt atttccatga gtttgacgaa cctgcggtga tctactcatc |
| 541 | caacggcaac aagaccgtgt ggcttcgtta cgcggagtgg ctgctgacct gccctgtcat |
| 601 | tcttatccat ctgagcaacc ttacgggtct ggcgaacgac tataacaagc gtaccatggg |
| 661 | tctgctggtg tcagatatcg gcacgatcgt gtggggcacc acggccgcgc tgtccaaggg |
| 721 | atacgtccgt gtcattttct tcctgatggg cctgtgctac ggcatctaca cattcttcaa |
| 781 | cgcagccaag gtctacattg aggcgtacca caccgtgccc aagggcattt gccgcgacct |
| 841 | ggtccgctac cttgcctggc tctacttctg ttcatgggct atgttcccgg tgctgttcct |
| 901 | gctgggcccc gagggctttg gccacatcaa ccaattcaac tctgccatcg cccacgccat |
| 961 | cctggacctt gcctccaaga acgcttggag tatgatgggt cactttctgc gtgtcaagat |
| 1021 | ccacgagcac atcctgctgt acggcgacat ccgcaagaag cagaaggtca acgtggctgg |
| 1081 | ccaggagatg gaggtggaga ccatggtgca cgaggaggac gacgagacgc agaaggtgcc |
| 1141 | cacggcaaag tacgccaacc gcgactcgtt catcatcatg cgcgaccgcc tcaaggagaa |
| 1201 | gggcttcgag acccgcgcct cgctggacgg cgacccgaac ggcgacgccg aggccaacgc |
| 1261 | tgcagccggc ggcaagcccg gaatggagat gggcaagatg accggcatgg gcatgggcat |
| 1321 | gggtgccggc atgggcatgg cgaccatcga ttcgggccgc gtcatcctcg ccgtgccgga |
| 1381 | catctccatg gtggactttt tccgcgagca gttcgcgcgg ctgcccgtgc cctacgaact |
| 1441 | ggtgcccgcg ctgggcgcgg agaacaccct ccagctggtg cagcaggcgc agtcactggg |
| 1501 | aggctgcgac ttcgtcctca tgcaccccga gttcctgcgc gaccgcagtc ccacgggtct |
| 1561 | gctgccccgc ctcaagatgg gcgggcagcg cgccgcggcc ttcggctggg cggcaatcgg |
| 1621 | ccccatgcgg gacttgatcg agggttcggg cgttgacggc tggctggagg gccccagctt |
| 1681 | tggcgccggc atcaaccagc aggcgctggt ggcgctgatc aaccgcatgc agcaggccaa |
| 1741 | gaagatgggc atgatgggcg gtatgggtat gggcatgggc ggcggcatgg gtatgggcat |
| 1801 | gggtatgggc atgggcatgg cccccagcat gaacgccggc atgactggcg gcatgggcgg |
| 1861 | cgcctccatg ggcggtgccg tgatgggcat gggcatgggc atgcagccca tgcagcaggc |
| 1921 | tatgccggcc atgtcgccca tgatgactca gcagcccagc atgatgagtc agccctccgc |
| 1981 | catgagcgcc ggcggcgcca tgcaggccat gggtggcgtc atgcccagcc ccgcccccgg |
| 2041 | cggccgcgtg ggcaccaacc cgctgtttgg ctctgcgccc tctccgctga gctcgcagcc |
| 2101 | cggcatcagc cctggcatgg cgacgccgcc cgccgccacc gccgcacccg ccgctggcgg |
| 2161 | cagcgaggcc gagatgctgc agcagctgat gagcgagatc aaccgcctga agaacgagct |
| 2221 | gggcgagtaa actgctggcc cagccgtacg gacatatgcc tgctgaggca ccagcgccgc |
| 2281 | aacacacatc gccgcagctg tcgcggctgc catgttggat ttgcgcgtgg cggcgtggtg |
| 2341 | gtgtggtggt gtggtggcag gaacaagggc gaagctttaa cttacccggc gctcagcgct |
| 2401 | tcgttcatag gttcggcgct tgagccgtgg tagcggcaag tgtgccgcgg caacgcgggg |
| 2461 | caaagcgaag acgccgatga cttgacgcct ggtatgacac cttggtctat gaagtcgcgc |
| 2521 | tgcggtgctg ggatcaagaa acagcaactc gaggaaggta tcatcgagcg tcgttataca |
| 2581 | gcagacaagg tacgaaacgg tgtgcaggag ggcatgcaca gcagcttcaa atggcacgtg |
| 2641 | catggctctg ttgcgaacaa gctgctctga gacacggatt gagagccctt aatcggtggt |
| 2701 | cacaagaggt ggggttacgg tatcggggcg ctgcgatagt cctgcaagtg ctgcctgttg |
| 2761 | aacacaaggg ctcagaattt atggcaggga aggtcaaggc cgagaatggc cgcgtgcgtg |
| 2821 | atttattgtt tgagccaggg cttgttgata ctgtattaat catgcgtgtg tgtttgtgtg |
| 2881 | cgtgaacgtg acccgacgga ttccgtgagc cgctgcgcat gcaagatccg gccctgacct |
| 2941 | atgtcctagt acaagccgat cgtgcttggc ctgccttgat taatgcgtcg cctgaggatt |
| 3001 | cccgtttgtg gcttttaagg agcgcgaata cggcagttac gtgacctgct tgtcgggttg |
| 3061 | gggaaatccg tctggtgtgt acctggcctg gccggctgat cgggtctgct tccggcaagt |
| 3121 | aactgtgcgg gtgaaactac aaaaggcagc gccggttgtg ggcgtcgttt tggttggttt |
| 3181 | ggcggggttc ccattgcaat gtgtgtttcc ataaatcatg ggcgacactg gatggaacgg |
| 3241 | ctttggcttg cgcggaggct tctcaggtcg gtacctaata ttgccataac ctctctttca |
| 3301 | aacctgcgcc tcctgcaatc aatagatgca gggggctgcg catcaaccct ggggaccata |
| 3361 | caatgcttaa ttccgctctg caattattcg agtagtggcc tgtcgcggag aagctgcttc |
| 3421 | agggtgtcaa tgtggctgca ggacggcaca ataaaagaga gtgtgggagc accgtatcct |
| 3481 | gaacagcggt ggattctcag agcctgtggg cgcttgcccg gcgcaccggc cgctcgtggg |
| 3541 | gggtagcagc tgcggctggt gtgctgatct tcatttgttt ctgtttgggg gggcacccct |
| 3601 | tgctctcgtt ggtgtgagcg ccggtgcgca gttgtaataa gggaagggag cataacgcgg |
| 3661 | cgtggcttac actaagagag ttgatacttt gaatcgacgc cttggatgca tgtaaaacca |
| 3721 | gaatttgaaa aaaaaaaaaa aaaaaaaaaa aaaaaaaaaa aaaaaaaaaa aaa |

The corresponding amino acid sequence encoding an exemplary Chop1 of the disclosure comprises or consists of GenBank accession number BAB68566:

| (SEQ ID NO: 8) | |
|---|---|
| 1 | msrrpwllal alavalaags agastgsdat vpvatqdgpd yvfhraherm lfqtsytlen |
| 61 | ngsvicipnn gqcfclawlk sngtnaekla anilqwitfa lsalclmfyg yqtwkstcgw |
| 121 | eeiyvatiem ikfiieyfhe fdepaviyss ngnktvwlry aewlltcpvi lihlsnitgl |
| 181 | andynkrtmg llvsdigtiv wgttaalskg yvrvifflmg lcygiytffn aakvyieayh |
| 241 | tvpkgicrdl vrylawlyfc swamfpvlfl lgpegfghin qfnsaiahai ldlasknaws |
| 301 | mmghflrvki hehillygdi rkkqkvnvag qemevetmvh eeddetqkvp takyanrdsf |
| 361 | iimrdrlkek gfetrasldg dpngdaeana aaggkpgmem gkmtgmgmgm gagmgmatid |
| 421 | sgrvilavpd ismvdffreq farlpvpyel vpalgaentl qlvqqaqslg gcdfvlmhpe |
| 481 | flrdrsptgl 1prlkmggqr aaafgwaaig pmrdliegsg vdgwlegpsf gaginqqalv |
| 541 | alinrmqqak kmgmmggmgm gmgggmgmgm gmgmgmapsm nagmtggmgg asmggavmgm |
| 601 | gmgmqpmqqa mpamspmmtq qpsmmsqpsa msaggamqam ggvmpspapg grvgtnplfg |
| 661 | sapsplssqp gispgmatpp aataapaagg seaemlqqlm seinrlknel ge |

A nucleic acid sequence encoding an exemplary Chop1 of the disclosure comprises or consists of GenBank accession number AF385748:

| (SEQ ID NO: 4) | |
|---|---|
| 1 | gcgttgcttg actacgcttc gctgtaataa tagcagcgcc acaagtagtg tcgccaaaca |
| 61 | actctcactt tgagcttgag cacaccgctg agccccgatg tcgcggaggc catggcttct |
| 121 | tgccctagcg ctggcagtgg cgctggcggc cggcagcgca ggagcctcga ctggcagtga |
| 181 | cgcgacggtg ccggtcgcga ctcaggatgg ccccgactac gttttccacc gtgcccacga |
| 241 | gcgcatgctc ttccaaacct catacactct tgagaacaat ggttctgtta tttgcatccc |
| 301 | gaacaacggc cagtgcttct gcttggcttg gcttaaatcc aacggaacaa atgccgagaa |
| 361 | gttggctgcc aacattctgc agtggattac ttttgcgctt tcagcgctct gcctgatgtt |
| 421 | ctacggctac cagacctgga agtctacttg cggctgggag gagatttacg tggccacgat |
| 481 | cgagatgatc aagttcatca tcgagtattt ccatgagttt gacgaacctg cggtgatcta |
| 541 | ctcatccaac ggcaacaaga ccgtgtggct tcgttacgcg gagtggctgc tgacctgccc |
| 601 | tgtcattctt atccatctga gcaaccttac gggtctggcg aacgactata acaagcgtac |
| 661 | catgggtctg ctggtgtcag atatcggcac gatcgtgtgg ggcaccacgg ccgcgctgtc |
| 21 | caagggatac gtccgtgtca ttttcttcct gatgggcctg tgctacggca tctacacatt |
| 781 | cttcaacgca gccaaggtct acattgaggc gtaccacacc gtgcccaagg gcatttgccg |
| 841 | cgacctggtc cgctaccttg cctggctcta cttctgttca tgggctatgt tcccggtgct |
| 901 | gttcctgctg ggccccgagg gctttggcca catcaaccaa ttcaactctg ccatcgccca |
| 961 | cgccatcctg gaccttgcct ccaagaacgc ttggagtatg atgggtcact ttctgcgtgt |
| 1021 | caagatccac gagcacatcc tgctgtacgg cgacatccgc aagaagcaga aggtcaacgt |
| 1081 | ggctggccag gagatggagg tggagaccat ggtgcacgag gaggacgacg agacgcagaa |
| 1141 | ggtgcccacg gcaaagtacg ccaaccgcga ctcgttcatc atcatgcgcg accgcctcaa |
| 1201 | ggagaagggc ttcgagaccc gcgcctcgct ggacggcgac ccgaacggcg acgccgaggc |
| 1261 | caacgctgca gccggcggca agcccggaat ggagatgggc aagatgaccg gcatgggcat |
| 1321 | gggcatgggt gccggcatgg gcatggcgac catcgattcg ggccgcgtca tcctcgccgt |
| 1381 | gccggacatc tccatggtgg actttttccg cgagcagttc gcgcggctgc ccgtgcccta |
| 1441 | cgaactggtg cccgcgctgg gcgcggagaa caccctccag ctggtgcagc aggcgcagtc |
| 1501 | actgggaggc tgcgacttcg tcctcatgca ccccgagttc ctgcgcgacc gcagtcccac |
| 1561 | gggtctgctg ccccgcctca agatgggcgg gcagcgcgcc gcggccttcg gctgggcggc |
| 1621 | aatcggcccc atgcgggact tgatcgaggg ttcgggcgtt gacggctggc tggagggccc |
| 1681 | cagctttggc gccggcatca accagcaggc gctggtggcg ctgatcaacc gcatgcagca |
| 1741 | ggccaagaag atgggcatga tgggcggtat gggtatgggc atgggcggcg gcatgggtat |
| 1801 | gggcatgggt atgggcatgg gcatggcccc cagcatgaac gccggcatga ctggcggcat |
| 1861 | gggcggcgcc tccatgggcg gtgccgtgat gggcatgggc atgggcatgc agcccatgca |
| 1921 | gcaggctatg ccggccatgt cgcccatgat gactcagcag cccagcatga tgagtcagcc |
| 1981 | ctccgccatg agcgccggcg gcgccatgca ggccatgggt ggcgtcatgc ccagccccgc |
| 2041 | ccccggcggc cgcgtgggca ccaacccgct gtttggctct gcgccctctc cgctgagctc |
| 2101 | gcagcccggc atcagccctg gcatggcgac gccgcccgcc gccaccgccg cacccgccgc |
| 2161 | tggcggcagc gaggccgaga tgctgcagca gctgatgagc gagatcaacc gcctgaagaa |
| 2221 | cgagctgggc gagtaa. |

The corresponding amino acid sequence encoding an exemplary Chop1 of the disclosure comprises or consists of GenBank accession number AAL08946.

| (SEQ ID NO: 9) | |
|---|---|
| 1 | msrrpwllal alavalaags agastgsdat vpvatqdgpd yvfhraherm lfqtsytlen |
| 61 | ngsvicipnn gqcfclawlk sngtnaekla anilqwitfa lsalclmfyg yqtwkstcgw |
| 121 | eeiyvatiem ikfiieyfhe fdepaviyss ngnktvwlry aewlltcpvi lihlsnitgl |
| 181 | andynkrtmg llvsdigtiv wgttaalskg yvrvifflmg lcygiytffn aakvyieayh |
| 241 | tvpkgicrdl vrylawlyfc swamfpvlfl lgpegfghin qfnsaiahai ldlasknaws |
| 301 | mmghflrvki hehillygdi rkkqkvnvag qemevetmvh eeddetqkvp takyanrdsf |
| 361 | iimrdrlkek gfetrasldg dpngdaeana aaggkpgmem gkmtgmgmgm gagmgmatid |
| 421 | sgrvilavpd ismvdffreq farlpvpyel vpalgaentl qlvqqaqslg gcdfvlmhpe |
| 481 | flrdrsptgl lprlkmggqr aaafgwaaig pmrdliegsg vdgwlegpsf gaginqqalv |
| 541 | alinrmqqak kmgmmggmgm gmgggmgmgm gmgmgmapsm nagmtggmgg asmggavmgm |
| 601 | gmgmqpmqqa mpamspmmtq qpsmmsqpsa msaggamqam ggvmpspapg grvgtnplfg |
| 661 | sapsplssqp gispgmatpp aataapaagg seaemlqqlm seinrlknel ge |

A nucleic acid sequence encoding an exemplary Chop1 of the disclosure comprises or consists of GenBank accession number AB058891:

| (SEQ ID NO: 5) | |
|---|---|
| 1 | catctgtcgc caagcaagca ttaaacatgg attatggagg cgccctgagt gccgttgggc |
| 61 | gcgagctgct atttgtaacg aacccagtag tcgtcaatgg ctctgtactt gtgcctgagg |
| 121 | accagtgtta ctgcgcgggc tggattgagt cgcgtggcac aaacggtgcc caaacggcgt |
| 181 | cgaacgtgct gcaatggctt gctgctggct tctccatcct actgcttatg ttttacgcct |
| 241 | accaaacatg gaagtcaacc tgcggctggg aggagatcta tgtgtgcgct atcgagatgg |
| 301 | tcaaggtgat tctcgagttc ttcttcgagt ttaagaaccc gtccatgctg tatctagcca |
| 361 | caggccaccg cgtccagtgg ttgcgttacg ccgagtggct tctcacctgc ccggtcattc |
| 421 | tcattcacct gtcaaacctg acgggcttgt ccaacgacta cagcaggcgc accatgggtc |
| 481 | tgcttgtgtc tgatattggc acaattgtgt ggggcgccac ttccgccatg gccaccggat |
| 541 | acgtcaaggt catcttcttc tgcctgggtc tgtgttatgg tgctaacacg ttctttcacg |
| 601 | ctgccaaggc ctacatcgag ggttaccaca ccgtgccgaa gggccggtgt cgccaggtgg |
| 661 | tgactggcat ggcttggctc ttcttcgtat catggggtat gttccccatc ctgttcatcc |
| 721 | tcggccccga gggcttcggc gtcctgagcg tgtacggctc caccgtcggc cacaccatca |
| 781 | ttgacctgat gtcgaagaac tgctggggtc tgctcggcca ctacctgcgc gtgctgatcc |
| 841 | acgagcatat cctcatccac ggcgacattc gcaagaccac caaattgaac attggtggca |
| 901 | ctgagattga ggtcgagacg ctggtggagg acgaggccga ggctggcgcg gtcaacaagg |
| 961 | gcaccggcaa gtacgcctcc cgcgagtcct tcctggtcat gcgcgacaag atgaaggaga |
| 1021 | agggcattga cgtgcgcgcc tctctggaca acagcaagga ggtggagcag gagcaggccg |
| 1081 | ccagggctgc catgatgatg atgaacggca atggcatggg tatgggaatg ggaatgaacg |
| 1141 | gcatgaacgg aatgggcggt atgaacggga tggctggcgg cgccaagccc ggcctggagc |
| 1201 | tcactccgca gctacagccc ggccgcgtca tcctggcggt gccggacatc agcatggttg |
| 1261 | acttcttccg cgagcagttt gctcagctat cggtgacgta cgagctggtg ccggccctgg |
| 1321 | gcgctgacaa cacactggcg ctggttacgc aggcgcagaa cctgggcggc gtggactttg |
| 1381 | tgttgattca ccccgagttc ctgcgcgacc gctctagcac cagcatcctg agccgcctgc |
| 1441 | gcggcgcggg ccagcgtgtg gctgcgttcg gctgggcgca gctggggccc atgcgtgacc |
| 1501 | tgatcgagtc cgcaaacctg gacggctggc tggagggccc ctcgttcgga cagggcatcc |
| 1561 | tgccggccca catcgttgcc ctggtggcca agatgcagca gatgcgcaag atgcagcaga |
| 1621 | tgcagcagat tggcatgatg accggcggca tgaacggcat gggcggcggt atgggcggcg |
| 1681 | gcatgaacgg catgggcggc ggcaacggca tgaacaacat gggcaacggc atgggcggcg |
| 1741 | gcatgggcaa cggcatgggc ggcaatggca tgaacggaat gggtggcggc aacggcatga |
| 1801 | acaacatggg cggcaacgga atggccggca acggaatggg cggcggcatg ggcggcaacg |
| 1861 | gtatgggtgg ctccatgaac ggcatgagct ccggcgtggt ggccaacgtg acgccctccg |
| 1921 | ccgccggcgg catgggcggc atgatgaacg gcggcatggc tgcgccccag tcgcccggca |
| 1981 | tgaacggcgg ccgcctgggt accaacccgc tcttcaacgc cgcgccctca ccgctcagct |
| 2041 | cgcagctcgg tgccgaggca ggcatgggca gcatgggagg catgggcgga atgagcggaa |
| 2101 | tgggaggcat gggtggaatg gggggcatgg gcggcgccgg cgccgccacg acgcaggctg |
| 2161 | cgggcggcaa cgcggaggcg gagatgctgc agaatctcat gaacgagatc aatcgcctga |
| 2221 | agcgcgagct tggcgagtaa aaggctggag gccggtactg cgatacctgc gagctcgcgc |
| 2281 | gcctgactcg tcgtacacac ggctcaggag cacgcgcgcg tggacttctc aacctgtgtg |
| 2341 | caacgtatct agagcggcct gtgcgcgacc gtccgtgagc attccggtgc gatcttcccg |
| 2401 | ccttcgcacc gcaagttccc ttcctggccc tgctgcgcct gacgcatc |

The corresponding amino acid sequence encoding an exemplary Chop1 of the disclosure comprises or consists of GenBank accession number BAB68567.1

| (SEQ ID NO: 10) | |
|---|---|
| 1 | mdyggalsav grellfvtnp vvvngsvlvp edqcycagwi esrgtngaqt asnvlqwlaa |
| 61 | gfsilllmfy ayqtwkstcg weeiyvcaie mvkvilefff efknpsmlyl atghrvqwlr |
| 121 | yaewlltcpv ilihlsnitg lsndysrrtm gllvsdigti vwgatsamat gyvkviffcl |
| 181 | glcygantff haakayiegy htvpkgrcrq vvtgmawlff vswgmfpilf ilgpegfgvl |
| 241 | svygstvght iidlmskncw glighylrvl ihehilihgd irkttklnig gteievetiv |
| 301 | edeaeagavn kgtgkyasre sflvmrdkmk ekgidvrasl dnskeveqeq aaraammmmn |
| 361 | gngmgmgmgm ngmngmggmn gmaggakpgl eltpqlqpgr vilavpdism vdffreqfaq |
| 421 | lsvtyelvpa lgadntlalv tqaqnlggvd fvlihpeflr drsstsilsr lrgagqrvaa |
| 481 | fgwaqlgpmr dliesanldg wlegpsfgqg ilpahivalv akmqqmrkmq qmqqigmmtg |
| 541 | gmngmgggmg ggmngmgggn gmnnmgngmg ggmgngmggn gmngmgggng mnnmggngma |
| 601 | gngmgggmgg ngmggsmngm ssgvvanvtp saaggmggmm nggmaapqsp gmnggrlgtn |
| 661 | plfnaapspl ssqlgaeagm gsmggmggms gmggmggmgg mggagaattq aaggnaeaem |
| 721 | lqnlmneinr lkrelge |

A nucleic acid sequence encoding an exemplary Chop1 of the disclosure comprises or consists of GenBank accession number AF461397:

| (SEQ ID NO: 6) | |
|---|---|
| 1 | gcatctgtcg ccaagcaagc attaaacatg gattatggag gcgccctgag tgccgttggg |
| 61 | cgcgagctgc tatttgtaac gaacccagta gtcgtcaatg gctctgtact tgtgcctgag |
| 121 | gaccagtgtt actgcgcggg ctggattgag tcgcgtggca caaacggtgc ccaaacggcg |
| 181 | tcgaacgtgc tgcaatggct tgctgctggc ttctccatcc tactgcttat gttttacgcc |
| 241 | taccaaacat ggaagtcaac ctgcggctgg gaggagatct atgtgtgcgc tatcgagatg |
| 301 | gtcaaggtga ttctcgagtt cttcttcgag tttaagaacc cgtccatgct gtatctagcc |
| 361 | acaggccacc gcgtccagtg gttgcgttac gccgagtggc ttctcacctg cccggtcatt |
| 421 | ctcattcacc tgtcaaacct gacgggcttg tccaacgact acagcaggcg caccatgggt |
| 481 | ctgcttgtgt ctgatattgg cacaattgtg tggggcgcca cttccgccat ggccaccgga |
| 541 | tacgtcaagg tcatcttctt ctgcctgggt ctgtgttatg gtgctaacac gttctttcac |
| 601 | gctgccaagg cctacatcga gggttaccac accgtgccga agggccggtg tcgccaggtg |
| 661 | gtgactggca tggcttggct cttcttcgta tcatggggta tgttccccat cctgttcatc |
| 721 | ctcggccccg agggcttcgg cgtcctgagc gtgtacggct ccaccgtcgg ccacaccatc |
| 781 | attgacctga tgtcgaagaa ctgctggggt ctgctcggcc actacctgcg cgtgctgatc |
| 841 | cacgagcata tcctcatcca cggcgacatt cgcaagacca ccaaattgaa cattggtggc |
| 901 | actgagattg aggtcgagac gctggtggag gacgaggccg aggctggcgc ggtcaacaag |
| 961 | ggcaccggca agtacgcctc ccgcgagtcc ttcctggtca tgcgcgacaa gatgaaggag |
| 1021 | aagggcattg acgtgcgcgc ctctctggac aacagcaagg aggtggagca ggagcaggcc |
| 1081 | gccagggctg ccatgatgat gatgaacggc aatggcatgg gtatgggaat gggaatgaac |
| 1141 | ggcatgaacg gaatgggcgg tatgaacggg atggctggcg gcgccaagcc cggcctggag |
| 1201 | ctcactccgc agctacagcc cggccgcgtc atcctggcgg tgccggacat cagcatggtt |
| 1261 | gacttcttcc gcgagcagtt tgctcagcta tcggtgacgt acgagctggt gccggccctg |
| 1321 | ggcgctgaca acacactggc gctggttacg caggcgcaga acctgggcgg cgtggacttt |
| 1381 | gtgttgattc accccgagtt cctgcgcgac cgctctagca ccagcatcct gagccgcctg |
| 1441 | cgcggcgcgg gccagcgtgt ggctgcgttc ggctgggcgc agctggggcc catgcgtgac |
| 1501 | ctgatcgagt ccgcaaacct ggacggctgg ctggagggcc cctcgttcgg acagggcatc |
| 1561 | ctgccggccc acatcgttgc cctggtggcc aagatgcagc agatgcgcaa gatgcagcag |
| 1621 | atgcagcaga ttggcatgat gaccggcggc atgaacggca tgggcggcgg tatgggcggc |
| 1681 | ggcatgaacg gcatgggcgg cggcaacggc atgaacaaca tgggcaacgg catgggcggc |
| 1741 | ggcatgggca acggcatggg cggcaatggc atgaacggaa tgggtggcgg caacggcatg |
| 1801 | aacaacatgg gcggcaacgg aatggccggc aacggaatgg gcggcggcat gggcggcaac |
| 1861 | ggtatgggtg gctccatgaa cggcatgagc tccggcgtgg tggccaacgt gacgccctcc |
| 1921 | gccgccggcg gcatgggcgg catgatgaac ggcggcatgg ctgcgcccca gtcgcccggc |
| 1981 | atgaacggcg gccgcctggg taccaacccg ctcttcaacg ccgcgccctc accgctcagc |
| 2041 | tcgcagctcg gtgccgaggc aggcatgggc agcatgggag gcatgggcgg aatgagcgga |
| 2101 | atgggaggca tgggtggaat ggggggcatg ggcggcgccg gcgccgccac gacgcaggct |
| 2161 | gcgggcggca acgcggaggc ggagatgctg cagaatctca tgaacgagat caatcgcctg |
| 2221 | aagcgcgagc ttggcgagta a |

The corresponding amino acid sequence encoding an exemplary Chop1 of the disclosure comprises or consists of GenBank accession number AAM15777.

| (SEQ ID NO: 11) | |
|---|---|
| 1 | mdyggalsav grellfvtnp vvvngsvlvp edqcycagwi esrgtngaqt asnvlqwlaa |
| 61 | gfsilllmfy ayqtwkstcg weeiyvcaie mvkvilefff efknpsmlyl atghrvqwlr |
| 121 | yaewlltcpv ilihlsnitg lsndysrrtm gllvsdigti vwgatsamat gyvkviffcl |
| 181 | glcygantff haakayiegy htvpkgrcrq vvtgmawlff vswgmfpilf ilgpegfgvl |
| 241 | svygstvght iidlmskncw glighylrvl ihehilihgd irkttklnig gteievetiv |
| 301 | edeaeagavn kgtgkyasre sflvmrdkmk ekgidvrasl dnskeveqeq aaraammmmn |
| 361 | gngmgmgmgm ngmngmggmn gmaggakpgl eltpqlqpgr vilavpdism vdffreqfaq |
| 421 | lsvtyelvpa lgadntlalv tqaqnlggvd fvlihpeflr drsstsilsr lrgagqrvaa |
| 481 | fgwaqlgpmr dliesanldg wlegpsfgqg ilpahivalv akmqqmrkmq qmqqigmmtg |
| 541 | gmngmgggmg ggmngmgggn gmnnmgngmg ggmgngmggn gmngmgggng mnnmggngma |
| 601 | gngmgggmgg ngmggsmngm ssgvvanvtp saaggmggmm nggmaapqsp gmnggrlgtn |
| 661 | plfnaapspl ssqlgaeagm gsmggmggms gmggmggmgg mggagaattq aaggnaeaem |
| 721 | lqnlmneinr lkrelge |

Channelopsins are a seven transmembrane domain proteins that become photo-switchable (light sensitive) when bound to the chromophore all-trans-retinal. Channelopsins, when linked to a retinal molecule via Schiff base linkage forms a light-gated, nonspecific, inwardly rectifying, cation channel, called a channelrhodopsin. These light-sensitive channels that, when expressed and activated in neural tissue, allow for a cell to be depolarized when stimulated with light (Boyden, 2005). A Chop2 fragment (315 amino acids) (SEQ ID NO: 7) has been shown to efficiently increase photosensitivity and vision in mouse models of photoreceptor degeneration (Bi et al., Neuron, 2006, and U.S. Pat. No. 8,470,790; both of which are hereby incorporated by reference).

Synthetic fragment of Chop2 protein, comprising 315 amino acids

(SEQ ID NO: 7)
MDYGGALSAVGRELLFVTNPVVVNGSVLVPEDQCYCAGWIESRGTNGAQT
ASNVLQWLAAGFSILLLMFYAYQTWKSTCGWEEIYVCAIEMVKVILEFFF
EFKNPSMLYLATGHRVQWLRYAEWLLTCPVILIHLSNLTGLSNDYSRRTM
GLLVSDIGTIVWGATSAMATGYVKVIFFCLGLCYGANTFFHAAKAYIEGY
HTVPKGRCRQVVTGMAWLFFVSWGMFPILFILGPEGFGVLSVYGSTVGHT
IIDLMSKNCWGLLGHYLRVLIHEHILIHGDIRKTTKLNIGGTEIEVETLV
EDEAEAGAVNKGTGK

Chop2 mutants and variants as described in PCT Publication WO 2013/134295 (hereby incorporated by reference) may also be expressed using the promoters described herein. The present invention also provides for use of Volvox carteri channelrhodopsins (i.e., vChR1 and vChR2).

NpHR (Halorhodopsin) (GenBank accession number EF474018) and (GenBank accession number AB064387) is from the haloalkaliphilic archaeon Natronomonas pharaonis. In certain embodiments variants of NpHR can be created. In specific embodiments single or multiple point mutations to the NpHR protein can result in NpHR variants. In specific embodiments a mammalian codon optimized version of NpHR can be utilized. In one embodiment NpHR variants are utilized. In one specific embodiment eNpHR (enhanced NpHR) is utilized. Addition of the amino acids FCYENEV to the NpHR C-terminus along with the signal peptide from the β subunit of the nicotinic acetylcholine receptor to the NpHR N-terminus results in the construction of eNpHR.

A nucleic acid sequence encoding an exemplary NpHR (Halorhodopsin) of the disclosure comprises or consists of GenBank accession number EF474018:

| (SEQ ID NO: 12) | |
|---|---|
| 1 | atgacagaga ccctgcctcc cgtgaccgag agtgccgtgg cccttcaagc cgaggttacc |
| 61 | caaagggagt tgttcgagtt cgtgctgaac gaccctttgc ttgcaagcag tctctatatc |
| 121 | aacatcgcac ttgcaggact gagtatactg ctgttcgttt ttatgacccg aggactcgat |
| 181 | gatccacggg caaaacttat tgctgtgtca accatccttg tgcctgtcgt cagcattgcc |
| 241 | tcctacactg gattggcgag cggcctgaca atttccgttc ttgaaatgcc agcgggccat |
| 301 | tttgcagaag gcagctcagt gatgctggga ggagaagagg tagatggtgt agtcaccatg |
| 361 | tggggacggt atctcacctg ggcactttcc acgcccatga ttctcctcgc tctgggtctc |
| 421 | ctggccggaa gcaatgctac aaagctcttc acagctatca ctttcgatat cgctatgtgc |
| 481 | gtgactggcc ttgccgcggc cctgactacc tcctcccacc tcatgagatg gttctggtac |
| 541 | gctatcagtt gtgcatgctt tctggtggtc ttgtatatcc tgctggtgga gtgggcacag |
| 601 | gacgccaaag ccgcgggaac cgctgacatg ttcaataccc tgaagctgtt gacagtagtg |
| 661 | atgtggctgg ggtatccaat tgtgtgggct cttggagtcg agggtatcgc ggtgttgccc |
| 721 | gttggggtga cgagctgggg atattctttc ctggatatcg tggcaaagta cattttcgca |
| 781 | ttcttgctcc tgaactatct gacgtcaaac gaatctgtcg tgtccggcag cattttggat |
| 841 | gttccatctg cttctgggac cccggctgat gattaa |

The corresponding amino acid sequence encoding an exemplary NpHR (Halorhodopsin) of the disclosure comprises or consists of GenBank accession number AB064387:

| (SEQ ID NO: 13) | |
|---|---|
| 1 | mtetlppvte savalqaevt grelfefvin dpllasslyi nialaglsil lfvfmtrgld |
| 61 | dprakliays tilvpvvsia sytglasglt isvlempagh faegssvmlg geevdgvvtm |
| 121 | wgryltwals tpmillalgl lagsnatklf taitfdiamc vtglaaaltt sshlmrwfwy |
| 181 | aiscacflvv lyillvewaq dakaagtadm fntlklltvv mwlgypivwa lgvegiavlp |
| 241 | vgvtswgysf ldivakyifa flllnyltsn esvvsgsild vpsasgtpad d |

Melanopsin (GenBank accession number 6693702) and (GenBank accession number AF147789_1) is a photopigment found in specialized photosensitive ganglion cells of the retina that are involved in the regulation of circadian rhythms, pupillary light reflex, and other non-visual responses to light. In structure, melanopsin is an opsin, a retinylidene protein variety of G-protein-coupled receptor. Melanopsin resembles invertebrate opsins in many respects, including its amino acid sequence and downstream signaling cascade. Like invertebrate opsins, melanopsin appears to be a bistable photopigment, with intrinsic photoisomerase activity. In certain embodiments variants of melanopsin can be created. In specific embodiments single or multiple point mutations to the melanopsin protein can result in melanopsin variants.

A nucleic acid sequence encoding an exemplary Melanopsin of the disclosure comprises or consists of GenBank accession number 6693702:

| (SEQ ID: 14) | |
|---|---|
| 1 | cactcattcc tttgcgcttc attggacatt aagcagtcag cagcccaaag agcagctcca |
| 61 | ggctggatgg atgagagcgg gcagcaggtg gaccaggccg cagggttaag gatggtatag |
| 121 | agccggaagt ctggggaccg atccctgatc tttccatggc cttagctcct ctgagagcct |
| 181 | gagcatggac tctccttcag gaccaagagt cttgtcaagc ttaactcagg atcccagctt |
| 241 | cacaaccagt cctgccctgc aaggcatttg gaacggcact cagaacgtct ccgtaagagc |
| 301 | ccagcttctc tctgttagcc ccacgacatc tgcacatcag gctgctgcct gggtcccctt |
| 361 | ccccacagtc gatgtcccag accatgctca ctatacccta ggcacggtga tcctgctggt |
| 421 | gggactcaca gggatgctgg gcaatctgac ggtcatctac accttctgca ggaacagagg |
| 481 | cctgcggaca ccagcaaaca tgttcatcat caacctcgca gtcagcgact tcctcatgtc |
| 541 | agtcactcag gccccggtct tctttgccag cagcctctac aagaagtggc tctttgggga |
| 601 | gacaggttgc gagttctatg ccttctgcgg ggctgtcttt ggcatcactt ccatgatcac |
| 661 | cctgacagcc atagccatgg accgctatct ggtgatcaca cgtccactgg ccaccatcgg |
| 721 | caggggatcc aaaagacgaa cggcactcgt cctgctaggc gtctggcttt atgccctggc |
| 781 | ctggagtctg ccacctttct ttggttggag tgcctacgtg cccgaggggc tgctgacatc |
| 841 | ctgctcctgg gactacatga ccttcacacc ccaggtgcgt gcctacacca tgctgctctt |
| 901 | ctgctttgtc ttcttcctcc ccctgctcat catcatcttc tgctacatct tcatcttcag |
| 961 | ggccatccga gagacaggcc gggcctgtga gggctgcggt gagtcccctc tgcggcagag |
| 1021 | gcggcagtgg cagcggctgc agagtgagtg gaagatggcc aaggtcgcac tgattgtcat |
| 1081 | tcttctcttc gtgctgtcct gggctcccta ctccactgtg gctctggtgg cctttgctgg |
| 1141 | atactcgcac atcctgacgc cctacatgag ctcggtgcca gccgtcatcg ccaaggcttc |
| 1201 | tgccatccac aatcccatta tctacgccat cactcacccc aagtacaggg tggccattgc |
| 1261 | ccagcacctg ccttgccttg gggtgcttct cggtgtatca ggccagcgca gccacccctc |
| 1321 | cctcagctac cgctctaccc accgctccac attgagcagc cagtcctcag acctcagctg |
| 1381 | gatctctgga cggaagcgtc aagagtccct gggttctgag agtgaagtgg gctggacaga |
| 1441 | cacagaaaca accgctgcat ggggagctgc ccagcaagca agtggacagt ccttctgcag |
| 1501 | tcagaaccta gaagatggag aactcaaggc ctcttccagc ccccaggtac agagatctaa |
| 1561 | gactcccaag gtgcctggac ccagtacctg ccgccctatg aaaggacagg gagccaggcc |
| 1621 | aagtagccta aggggtgacc agaaaggcag gcttgctgtg tgcacaggcc tctcagagtg |
| 1681 | tccccatccc catacatccc agtttcccct tgctttccta gaggatgatg tgactctcag |
| 1741 | acatctgtag cagggtctaa gtatgatctg tatctagggg aatatctgca tgtgactgtg |
| 1801 | tagctctgcg catgacatgc tgtcagctat gttgtaccat atgtatatgt agagtatgca |
| 1861 | tataacttat gtgcccttga agatatgtgg cctacagcag agaacaactc atgcgtgtgt |
| 1921 | ggaccatgtt cctggcatat atgctctctg tcactgtgat gcctctgtgt tgtgtgggtg |
| 1981 | acagagtgtg atggtgttca cctctctgcg cgggttttga tgctgggcaa acacggggaa |
| 2041 | gggagctgca agccatgtac tagctcactg ccgatggcct gtgctcaaga tgtcaccgag |
| 2101 | gagaacactt gtagctatta aaagaaggcc agctgtc |

The corresponding amino acid sequence encoding an exemplary Melanopsin of the disclosure comprises or consists of GenBank accession number AF1477891:

| | (SEQ ID NO: 15) |
|---|---|
| 1 | mdspsgprvl ssltqdpsft tspalqgiwn gtqnvsvraq llsyspttsa hqaaawvpfp |
| 61 | ttvdvpdhah ylgtvillvg ltgmlgnitv iytfcrnrgl rtpanmfiin laysdflmsv |
| 121 | tqapvffass lykkwlfget gcefyafcga vfgitsmitl taiamdrylv itrplatigr |
| 181 | gskrrtalvl lgvwlyalaw slppffgwsa yvpeglltsc swdymtftpq vraytmllfc |
| 241 | fvfflpllii ifcyififra iretgraceg cgesplrqrr qwqrlqsewk makvalivil |
| 301 | lfvlswapys tvalvafagy shiltpymss vpaviakasa ihnpiiyait hpkyrvaiaq |
| 361 | hlpclgvllg vsgqrshpsl syrsthrstl ssqssdlswi sgrkrqeslg sesevgwtdt |
| 421 | ettaawgaaq qasgqsfcsq nledgelkas sspqvqrskt pkvpgpstcr pmkgqgarps |
| 481 | slrgdqkgrl avctglsecp hphtsqfpla fleddvtlrh l. |

Light-sensitive proteins may also include proteins that are at least about 10%, at least about 20%, at least about 25%, at least about 30%, at least about 35%, at least about 40%, at least about 45%, at least about 50%, at least about 55%, at least about 60%, at least about 70%, at least about 80%, at least about 90%, at least about 95%, or at least about 99% identical to any of the light-sensitive proteins described herein (i.e., ChR1, ChR2, vChR1, vChR2, NpHR and melanopsin). The light-sensitive proteins of the present invention may also include proteins that have at least one mutation. The mutation may be a point mutation.

In some embodiments, light-sensitive proteins can modulate signaling within neural circuits and bidirectionally control behavior of ionic conductance at the level of a single neuron. In some embodiments the neuron is a retinal neuron, a retinal bipolar cell (e.g. ON or OFF retinal bipolar cells; rod and cone bipolar cells), a retinal ganglion cell, a photoreceptor cell, or a retinal amacrine cell.

In some embodiments, a polyA tail can be inserted downstream of the transgene in an expression cassette or nucleic acid expression vector of the present invention. Suitable polyA tails are known in the art, and include, for example, human growth hormone poly A tail (hGHpA), bovine growth hormone polyA tail (bGHpA), bovine polyA, SV40 polyA, and AV40pA.

Upon illumination by the preferred dose of light radiation, rhodopsin proteins opens the pore of the channel, through which H⁺, Na⁺, K⁺, and/or Ca²⁺ ions flow into the cell from the extracellular space. Activation of the rhodopsin channel typically causes a depolarization of the cell expressing the channel. Depolarized cells produce graded potentials and or action potentials to carry information from the rhodopsin-expressing cell to other cells of the retina or brain, to increase light sensitivity or restore vision. Methods of improving vision or light sensitivity by administration of a vector encoding a channelopsin (or variant thereof) are described in PCT/US2007/068263, the contents of which are herein incorporated in its entirety.

Accordingly, a dual rhodopsin system can be used to recapitulate the ON and OFF pathways integral to visual processing and acuity. Briefly, a Chop2 protein of the present invention can be specifically targeted to ON type retinal neurons (i.e., ON type ganglion cells and/or ON type bipolar cells), while a hypopolarizing light sensor (i.e., halorhodopsin or other chloride pump known in the art) can be targeted to OFF type retinal neurons (i.e. OFF type ganglion cells and/or OFF type bipolar cells) to create ON and OFF pathways. The specific targeting to preferred cell subpopulations can be achieved through the use of different cell type-specific promoters. For example, Chop2 expression may be driven by the mGluR6 promoter for targeted expression in ON-type retinal neurons (i.e., ON type ganglion cells and/or ON type bipolar cells) while a hypopolarizing channel, such as halorhodopsin, expression is driven by the NK-3 promoter for targeted expression in OFF-type retinal neurons (i.e., OFF type ganglion cells and/or OFF type bipolar cells).

An alternative approach to restore ON and OFF pathways in the retina is achieved by, expressing a depolarizing light sensor, such as ChR2, to rod bipolar cells or All amacrine. In this approach, the depolarization of rod bipolar cells or AII amacrine cells can lead to the ON and OFF responses at the levels of cone bipolar cells and the downstream retinal ganglion cells. Thus, the ON and OFF pathways that are inherent in the retina are maintained.

An effective amount of rAAV virions carrying a nucleic acid sequence encoding the rhodopsin DNA under the control of the promoter of choice, preferably a constitutive CMV promoter or a cell-specific promoter such as mGluR6, is preferably in the range of between about 10¹⁰ to about 10¹³ rAAV infectious units in a volume of between about 25 and about 800 μl per injection. The rAAV infectious units can be measured according to McLaughlin, S K et al., 1988, J Virol 62:1963. More preferably, the effective amount is between about 10¹⁰ and about 10¹² rAAV infectious units and the injection volume is preferably between about 50 and about 150 μl. Other dosages and volumes, preferably within these ranges but possibly outside them, may be selected by the treating professional, taking into account the physical state of the subject (preferably a human), who is being treated, including, age, weight, general health, and the nature and severity of the particular ocular disorder.

It may also be desirable to administer additional doses (“boosters”) of the present nucleic acid(s) or rAAV compositions. For example, depending upon the duration of the transgene expression within the ocular target cell, a second treatment may be administered after 6 months or yearly, and may be similarly repeated. Neutralizing antibodies to AAV are not expected to be generated in view of the routes and doses used, thereby permitting repeat treatment rounds.

The need for such additional doses can be monitored by the treating professional using, for example, well-known electrophysiological and other retinal and visual function tests and visual behavior tests. The treating professional will be able to select the appropriate tests applying routine skill in the art. It may be desirable to inject larger volumes of the composition in either single or multiple doses to further improve the relevant outcome parameters.

### Ocular Disorders

The ocular disorders for which the methods of the present invention are intended and may be used to improve one or more parameters of vision include, but are not limited to, developmental abnormalities that affect both anterior and posterior segments of the eye. Anterior segment disorders include glaucoma, cataracts, corneal dystrophy, keratoconus. Posterior segment disorders include blinding disorders caused by photoreceptor malfunction and/or death caused by retinal dystrophies and degenerations. Retinal disorders include congenital stationary night blindness, age-related macular degeneration, congenital cone dystrophies, and a large group of retinitis-pigmentosa (RP)-related disorders. These disorders include genetically pre-disposed death of photoreceptor cells, rods and cones in the retina, occurring at various ages. Among those are severe retinopathies, such as subtypes of RP itself that progresses with age and causes blindness in childhood and early adulthood and RP-associated diseases, such as genetic subtypes of LCA, which frequently results in loss of vision during childhood, as early as the first year of life. The latter disorders are generally characterized by severe reduction, and often complete loss of photoreceptor cells, rods and cones. Other ocular diseases that may benefit from the methods described herein include, but are not limited to, retinoblastoma, ocular melanoma, diabetic retinopathy, hypertensive retinopathy, any inflammation of the ocular tissues (i.e., chorioretinal inflammation, scleritis, keratitis, uveitis, etc.), or infection (i.e., bacterial or viral).

In particular, the viral-mediated delivery of rhodopsins using the methods of the present invention useful for the treatment and/or restoration of at least partial vision to subjects that have lost vision due to ocular disorders, such as RPE-associated retinopathies, which are characterized by a long-term preservation of ocular tissue structure despite loss of function and by the association between function loss and the defect or absence of a normal gene in the ocular cells of the subject. A variety of such ocular disorders are known, such as childhood onset blinding diseases, retinitis pigmentosa, macular degeneration, and diabetic retinopathy, as well as ocular blinding diseases known in the art. It is anticipated that these other disorders, as well as blinding disorders of presently unknown causation which later are characterized by the same description as above, may also be successfully treated by the methods described herein. Thus, the particular ocular disorder treated by the present invention may include the above-mentioned disorders and a number of diseases which have yet to be so characterized.

### Restoration of Light Sensitivity

These methods described herein may be used in subjects of normal and/or impaired vision. The enhanced delivery of a therapeutic compound, as described herein, may preserve, improve, or restore vision. The term “vision” as used herein is defined as the ability of an organism to usefully detect light as a stimulus for differentiation or action. Vision is intended to encompass the following:
1. Light detection or perception—the ability to discern whether or not light is present;
2. Light projection—the ability to discern the direction from which a light stimulus is coming;
3. Resolution—the ability to detect differing brightness levels (i.e., contrast) in a grating or letter target; and
4. Recognition—the ability to recognize the shape of a visual target by reference to the differing contrast levels within the target.
   Thus, “vision” includes the ability to simply detect the presence of light. The methods of the present invention can be used to improve or restore vision, wherein the improvement or restoration in vision includes, for example, increases in light detection or perception, increase in light sensitivity or photosensitivity in response to a light stimulus, increase in the ability to discern the direction from which a light stimulus is coming, increase in the ability to detect differing brightness levels, increase in the ability to recognize the shape of a visual target, and increases in visual evoked potential or transmission from the retina to the cortex. As such, improvement or restoration of vision may or may not include full restoration of sight, i.e., wherein the vision of the patient treated with the present invention is restored to the degree to the vision of a non-affected individual. The visual recovery described in the animal studies described below may, in human terms, place the person on the low end of vision function by increasing one aspect of vision (i.e., light sensitivity, or visual evoked potential) without restoring full sight. Nevertheless, placement at such a level would be a significant benefit because these individuals could be trained in mobility and potentially in low order resolution tasks which would provide them with a greatly improved level of visual independence compared to total blindness. Even basic light perception can be used by visually impaired individuals, whose vision is improved using the present compositions and methods, to accomplish specific daily tasks and improve general mobility, capability, and quality of life.

The degree of restoration of vision can be determined through the measurement of vision before, and preferably after, administering a vector comprising, for example, DNA encoding a therapeutic transgene such as Chop2 or halorhodopsin or both. Vision can be measured using any of a number of methods well-known in the art or methods not yet established. Vision, as improved or restored by the present invention, can be measured by any of the following visual responses:
1. a light detection response by the subject after exposure to a light stimulus—in which evidence is sought for a reliable response of an indication or movement in the general direction of the light by the subject individual when the light it is turned on;
2. a light projection response by the subject after exposure to a light stimulus in which evidence is sought for a reliable response of indication or movement in the specific direction of the light by the individual when the light is turned on;
3. light resolution by the subject of a light vs. dark patterned visual stimulus, which measures the subject's capability of resolving light vs dark patterned visual stimuli as evidenced by:
   a. the presence of demonstrable reliable optokinetically produced nystagmoid eye movements and/or related head or body movements that demonstrate tracking of the target (see above) and/or
   b. the presence of a reliable ability to discriminate a pattern visual stimulus and to indicate such discrimination by verbal or non-verbal means, including, for example pointing, or pressing a bar or a button; or
4. electrical recording of a visual cortex response to a light flash stimulus or a pattern visual stimulus, which is an endpoint of electrical transmission from a restored retina to the visual cortex, also referred to as the visual evoked potential (VEP). Measurement may be by electrical recording on the scalp surface at the region of the visual cortex, on the cortical surface, and/or recording within cells of the visual cortex.

Thus, improvement or restoration of vision, according to the present invention, can include, but is not limited to: increases in amplitude or kinetics of photocurents or electrical response in response to light stimulus in the retinal cells, increases in light sensitivity (i.e., lowering the threshold light intensity required for initiating a photocurrent or electrical response in response to light stimulus, thereby requiring less or lower light to evoke a photocurrent) of the retinal cells, increases in number or amplitude of light-evoked spiking or spike firings, increases in light responses to the visual cortex, which includes increasing in visual evoked potential transmitted from the retina or retinal cells to the visual cortex or the brain.

Both in vitro and in vivo studies to assess the various parameters of the present invention may be used, including recognized animal models of blinding human ocular disorders. Large animal models of human retinopathy, e.g., childhood blindness, are useful. The examples provided herein allow one of skill in the art to readily anticipate that this method may be similarly used in treating a range of retinal diseases.

While earlier studies by others have demonstrated that retinal degeneration can be retarded by gene therapy techniques, the present invention demonstrates a definite physiological recovery of function, which is expected to generate or improve various parameters of vision, including behavioral parameters.

Behavioral measures can be obtained using known animal models and tests, for example performance in a water maze, wherein a subject in whom vision has been preserved or restored to varying extents will swim toward light (Hayes, J M et al., 1993, Behav Genet 23:395-403).

In models in which blindness is induced during adult life or congenital blindness develops slowly enough that the individual experiences vision before losing it, training of the subject in various tests may be done. In this way, when these tests are re-administered after visual loss to test the efficacy of the present compositions and methods for their vision-restorative effects, animals do not have to learn the tasks de novo while in a blind state. Other behavioral tests do not require learning and rely on the instinctiveness of certain behaviors. An example is the optokinetic nystagmus test (Balkema G W et al., 1984, Invest Ophthalmol Vis Sci. 25:795-800; Mitchiner J C et al., 1976, Vision Res. 16:1169-71).

The present invention may also be used in combination with other forms of vision therapy known in the art to improve or restore vision. For example, the use of visual prostheses, which include retinal implants, cortical implants, lateral geniculate nucleus implants, or optic nerve implants. Thus, in addition to genetic modification of surviving retinal neurons using the present methods, the subject being treated may be provided with a visual prosthesis before, at the same time as, or after the molecular method is employed. The effectiveness of visual prosthetics can be improved with training of the individual, thus enhancing the potential impact of the Chop2 transformation of patient cells as contemplated herein. Training methods, such as habituation training characterized by training the subject to recognize (i) varying levels of light and/or pattern stimulation, and/or (ii) environmental stimulation from a common light source or object as would be understood by one skilled in the art; and orientation and mobility training characterized by training the subject to detect visually local objects and move among said objects more effectively than without the training. In fact, any visual stimulation techniques that are typically used in the field of low vision rehabilitation are applicable here.

### Definitions

Unless defined otherwise, all technical and scientific terms used herein have the same meaning as commonly understood by those of ordinary skill in the art to which the invention belongs. For the purposes of the present invention, the following terms are defined below.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “adeno-associated viral vector” refers to a viral vector or plasmid containing structural and functional genetic elements, or portions thereof, that are primarily derived from a adenovirus The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus. The term “hybrid” refers to a vector, LTR or other nucleic acid containing both viral and non-viral viral sequences.

In particular aspects, the terms “viral vector,” “viral expression vector” may be used to refer to viral transfer plasmids and/or infectious viral particles. Where reference is made herein to elements such as cloning sites, promoters, regulatory elements, heterologous nucleic acids, etc., it is to be understood that the sequences of these elements are present in RNA form in the viral particles of the invention and are present in DNA form in the DNA plasmids of the invention.

At each end of the provirus are structures called “long terminal repeats” or “LTRs.” The term “long terminal repeat (LTR)” refers to domains of base pairs located at the ends of retroviral DNAs which, in their natural sequence context, are direct repeats and contain U3, R and U5 regions. LTRs generally provide functions fundamental to the expression of viral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR composed of U3, R and U5 regions and appears at both the 5′ and 3′ ends of the viral genome. Adjacent to the 5′ LTR are sequences necessary for reverse transcription of the genome (the tRNA primer binding site) and for efficient packaging of viral RNA into particles (the Psi site).

As used herein, the term “packaging signal” or “packaging sequence” refers to sequences located within the viral genome which are required for insertion of the viral RNA into the viral capsid or particle, see e.g., Clever et al., 1995. J. of Virology, Vol. 69, No. 4; pp. 2101-2109. As used herein, the terms “packaging sequence,” “packaging signal,” “psi” and the symbol “‘PSI,” are used in reference to the non-coding sequence required for encapsidation of retroviral RNA strands during viral particle formation.

In various aspects, vectors comprise modified 5′ LTR and/or 3′ LTRs. Modifications of the 3′ LTR are often made to improve the safety of the viral systems by rendering viruses replication-defective. As used herein, the term “replication-defective” refers to virus that is not capable of complete, effective replication such that infective virions are not produced (e.g., replication-defective lentiviral progeny). The term “replication-competent” refers to wild-type virus or mutant virus that is capable of replication, such that viral replication of the virus is capable of producing infective virions (e.g., replication-competent lentiviral progeny).

“Self-inactivating” (SIN) vectors refers to replication-defective vectors, in which the right (3′) LTR enhancer-promoter region, known as the U3 region, has been modified (e.g., by deletion and/or substitution) to prevent viral transcription beyond the first round of viral replication. This is because the right (3′) LTR U3 region is used as a template for the left (5′) LTR U3 region during viral replication and, thus, the viral transcript cannot be made without the U3 enhancer-promoter. In a further aspect of the invention, the 3′ LTR is modified such that the U5 region is replaced, for example, with a heterologous or synthetic poly(A) sequence, one or more insulator elements, and/or an inducible promoter. It should be noted that modifications to the LTRs such as modifications to the 3′ LTR, the 5′ LTR, or both 3′ and 5′ LTRs, are also included in the invention.

An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In certain aspects, the heterologous promoter may be inducible, such that transcription of all or part of the viral genome will occur only when one or more induction factors are present. Induction factors include, but are not limited to, one or more chemical compounds or physiological conditions, e.g., temperature or pH, in which the host cells are cultured.

In some aspects, viral vectors comprise a TAR element. The term “TAR” refers to the “trans-activation response” genetic element located in the R region of lentiviral (e.g., HIV) LTRs. This element interacts with the viral trans-activator (tat) genetic element to enhance viral replication. However, this element is not required in aspects wherein the U3 region of the 5′ LTR is replaced by a heterologous promoter.

As used herein, the term “FLAP element” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173. During HIV-1 reverse transcription, central initiation of the plus-strand DNA at the central polypurine tract (cPPT) and central termination at the central termination sequence (CTS) lead to the formation of a three-stranded DNA structure: the HIV-1 central DNA flap. While not wishing to be bound by any theory, the DNA flap may act as a cis-active determinant of lentiviral genome nuclear import and/or may increase the titer of the virus. In particular aspects, the retroviral or lentiviral vector backbones comprise one or more FLAP elements upstream or downstream of the heterologous genes of interest in the vectors. For example, in particular aspects a transfer plasmid includes a FLAP element. In one aspect, a vector of the invention comprises a FLAP element isolated from HIV-1.

In one aspect, viral transfer vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE). Generally, the RNA export element is placed within the 3′ UTR of a gene, and can be inserted as one or multiple copies.

In particular aspects, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766). In particular aspects, vectors of the invention lack or do not comprise a posttranscriptional regulatory element such as a WPRE or HPRE because in some instances these elements increase the risk of cellular transformation and/or do not substantially or significantly increase the amount of mRNA transcript or increase mRNA stability. Therefore, in some aspects, vectors of the invention lack or do not comprise a WPRE or HPRE as an added safety measure.

Elements directing the efficient termination and polyadenylation of the heterologous nucleic acid transcripts increases heterologous gene expression. Transcription termination signals are generally found downstream of the polyadenylation signal. The term “polyA site” or “polyA sequence” as used herein denotes a DNA sequence which directs both the termination and polyadenylation of the nascent RNA transcript by RNA polymerase II. Efficient polyadenylation of the recombinant transcript is desirable as transcripts lacking a poly A tail are unstable and are rapidly degraded. Illustrative examples of polyA signals that can be used in a vector of the invention, include an ideal polyA sequence (e.g., AATAAA, ATTAAA AGTAAA), a bovine growth hormone polyA sequence (BGHpA), a rabbit .beta.-globin polyA sequence (r.beta.gpA), or another suitable heterologous or endogenous polyA sequence known in the art.

In certain aspects, viral vector further comprises one or more insulator elements. Insulators elements may contribute to protecting lentivirus-expressed sequences, e.g., therapeutic polypeptides, from integration site effects, which may be mediated by cis-acting elements present in genomic DNA and lead to deregulated expression of transferred sequences (i.e., position effect; see, e.g., Burgess-Beusse et al., 2002, Proc. Natl. Acad. Sci., USA, 99:16433; and Zhan et al., 2001, Hum. Genet., 109:471). In some aspects, transfer vectors comprise one or more insulator element the 3′ LTR and upon integration of the provirus into the host genome, the provirus comprises the one or more insulators at both the 5′ LTR or 3′ LTR, by virtue of duplicating the 3′ LTR. Suitable insulators for use in the invention include, but are not limited to, the chicken .beta.-globin insulator (see, e.g., Chung et al., 1993. Cell 74:505; Chung et al., 1997. PNAS 94:575; and Bell et al., 1999. Cell 98:387, incorporated by reference herein). Examples of insulator elements include, but are not limited to, an insulator from an .beta.-globin locus, such as chicken HS4.

As used herein, the term “time sufficient to increase transduction efficiency” refers to a time period in which a population of cells may be cultured together with a proteasome inhibitor, when the population of cells is brought into contact with a gene delivery vehicle, such as an adenovirus, the cells are transduced with the gene delivery vehicle at a higher transduction efficiency, defined as the percentage of cells which are transduced with the gene delivery vehicle, compared to a similar population of cells that is brought into contact with a similar gene delivery vehicle, in the absence a proteasome inhibitor.

As used herein, the term “transduction efficiency” refers to the percentage of cells cultured with a compound that increases prostaglandin signaling that are transduced with a gene delivery vehicle, compared to a similar population of cells that is brought into contact with a similar gene delivery vehicle, in the absence of the compound that increases

A “small molecule,” “small organic molecule,” or “small molecule compound” refers to a low molecular weight compound that has a molecular weight of less than about 5 kD, less than about 4 kD, less than about 3 kD, less than about 2 kD, less than about 1 kD, or less than about 0.5 kD. In particular aspects, small molecules can include, nucleic acids, peptides, peptidomimetics, peptoids, other small organic compounds or drugs, and the like. Libraries of chemical and/or biological mixtures, such as fungal, bacterial, or algal extracts, are known in the art and can be screened with any of the assays of the invention. Examples of methods for the synthesis of molecular libraries can be found in: (Carell et al., 1994a; Carell et al., 1994b; Cho et al., 1993; DeWitt et al., 1993; Gallop et al., 1994; Zuckermann et al., 1994).

As used herein, the terms “polynucleotide” or “nucleic acid” refers to messenger RNA (mRNA), RNA, genomic RNA (gRNA), plus strand RNA (RNA(+)), minus strand RNA (RNA(−)), genomic DNA (gDNA), complementary DNA (cDNA) or DNA. Polynucleotides include single and double stranded polynucleotides. Preferably, polynucleotides of the invention include polynucleotides or variants having at least about 50%, 55%, 60%, 65%, 70%, 75%, 80%, 85%, 90%, 95%, 96%, 97%, 98%, 99% or 100% sequence identity to any of the reference sequences described herein (see, e.g., Sequence Listing), typically where the variant maintains at least one biological activity of the reference sequence. In various illustrative aspects, the present invention contemplates, in part, viral vector and transfer plasmid polynucleotide sequences and compositions comprising the same. In particular aspects, the invention provides polynucleotides encoding one or more therapeutic polypeptides and/or other genes of interest.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted, or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

As used herein, the term “isolated” means material, e.g., a polynucleotide, a polypeptide, a cell, that is substantially or essentially free from components that normally accompany it in its native state. In particular aspects, the term “obtained” or “derived” is used synonymously with isolated. For example, an “isolated polynucleotide,” as used herein, refers to a polynucleotide that has been purified from the sequences which flank it in a naturally-occurring state, e.g., a DNA fragment that has been removed from the sequences that are normally adjacent to the fragment.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation.

The terms “complementary” and “complementarity” refer to polynucleotides (i.e., a sequence of nucleotides) related by the base-pairing rules. For example, the complementary strand of the DNA sequence 5′ A G T C A T G 3′ is 3′ T C A G T A C5′. The latter sequence is often written as the reverse complement with the 5′ end on the left and the 3′ end on the right, 5′ CAT GAC T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

The term “nucleic acid cassette” as used herein refers to genetic sequences within the vector which can express an RNA, and subsequently a polypeptide. In one aspect, the nucleic acid cassette contains a gene(s)-of-interest, e.g., a polynucleotide(s)-of-interest. In another aspect, the nucleic acid cassette contains one or more expression control sequences and a gene(s)-of-interest, e.g., a polynucleotide(s)-of-interest. Vectors may comprise one, two, three, four, five or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. In a preferred aspect of the invention, the nucleic acid cassette contains the sequence of a therapeutic gene used to treat, prevent, or ameliorate a genetic disorder, such as an ocular disorder. The cassette can be removed and inserted into a plasmid or viral vector as a single unit.

Polynucleotides include a polynucleotide(s)-of-interest. As used herein, the term “polynucleotide(s)-of-interest” refers to one or more polynucleotides, e.g., a polynucleotide encoding a polypeptide (i.e., a polypeptide-of-interest), inserted into an expression vector th

The term “expression control sequence” refers to a polynucleotide sequence that comprises one or more promoters, enhancers, or other transcriptional control elements or combinations thereof that are capable of directing, increasing, regulating, or controlling the transcription or expression of an operatively linked polynucleotide. In particular aspects, vectors of the invention comprise one or more expression control sequences that are specific to particular cells, cell types, or cell lineages e.g., target cells; that is, expression of polynucleotides operatively linked to an expression control sequence specific to particular cells, cell types, or cell lineages is expressed in target cells and not in other non-target cells. Each one of the one or more expression control sequences in a vector that are cell specific may express in the same or different cell types depending on the therapy desired. In preferred aspects, vectors comprise one or more expression control sequences specific to hematopoietic cells, e.g., hematopoietic stem or progenitor cells. In other preferred aspects, vectors comprise one or more expression control sequences specific to erythroid cells.

The term “promoter” as used herein refers to a recognition site of a polynucleotide (DNA or RNA) to which an RNA polymerase binds. The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

In particular aspects, a vector of the invention comprises exogenous, endogenous, or heterologous control sequences such as promoters and/or enhancers. An “endogenous” control sequence is one which is naturally linked to a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated. A “synthetic” control sequence may comprise elements of one more endogenous and/or exogenous sequences, and/or sequences determined in vitro or in silico that provide optimal promoter and/or enhancer activity for the particular gene therapy.

The term “operably linked”, refers to a juxtaposition wherein the components described are in a relationship permitting them to function in their intended manner. In one aspect, the term refers to a functional linkage between a nucleic acid expression control sequence (such as a promoter, and/or enhancer or other expression control sequence) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively. Illustrative ubiquitous expression control sequences include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), .beta.-kinesin (.beta.-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken .beta.-actin (CAG) promoter, and a .beta.-actin promoter.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain aspects of the invention provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Conditional expression can also be achieved by using a site specific DNA recombinase. According to certain aspects of the invention the vector comprises at least one (typically two) site(s) for recombination mediated by a site specific recombinase. As used herein, the terms “recombinase” or “site specific recombinase” include excisive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use in particular aspects of the present invention include, but are not limited to: Cre, Int, IHF, Xis, Flp, Fis, Hin, Gin, .PHI.C31, Cin, Tn3 resolvase, TndX, XerC, XerD, TnpX, Hjc, Gin, SpCCE1, and ParA.

The vectors may comprise one or more recombination sites for any of a wide variety of site specific recombinases. It is to be understood that the target site for a site specific recombinase is in addition to any site(s) required for integration of a vector. As used herein, the terms “recombination sequence,” “recombination site,” or “site specific recombination site” refer to a particular nucleic acid sequence to which a recombinase recognizes and binds.

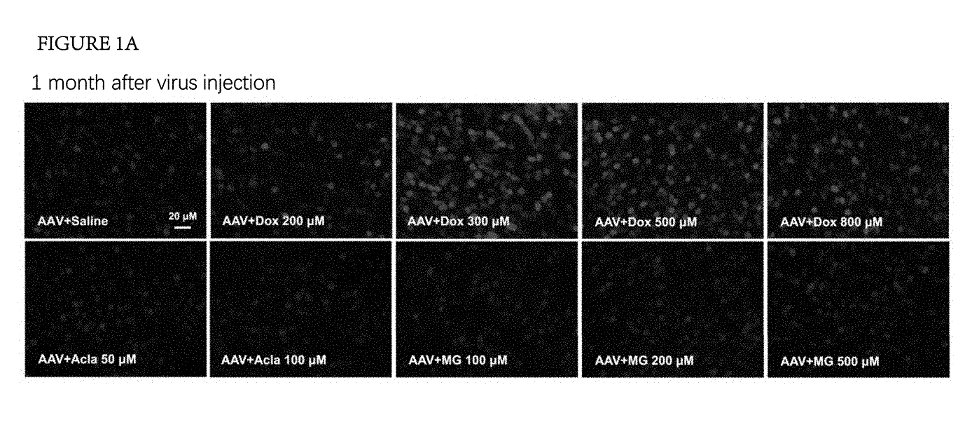

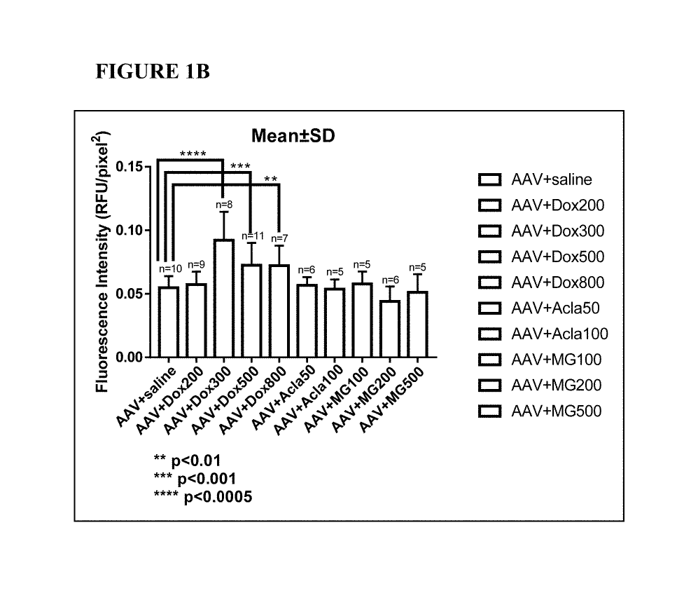

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)) Other exemplary loxP sites include, but are not limited to: lox511, (Hoess et al., Nucleic Acids Res. 14: 2287-2300, 1996; Bethke and Sauer, Nucleic Acids Res; 25: 2828-2834, 1997); lox5171, (Lee and Saito, Gene. 216: 55-65, 1998); lox2272 (Lee and Saito, Gene. 216: 55-65, 1998); m2 (Langer et al., Nucleic Acids Res. 30: 3067-3077, 2002), lox71 (Albert et al., Plant J.; 7: 649-659, 1995), and lox66, (Albert et al., Plant J.; 7: 649-659, 1995).

Suitable recognition sites for the FLP recombinase include, but are not limited to: FRT (McLeod, et al., 1996), F1, F2, F3 (Schlake and Bode, 1994), F4, F5 (Schlake and Bode, 1994), FRT(LE), (Senecoff et al., 1988) and FRT(RE), (Senecoff et al., 1988).

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. In particular aspects, the vectors contemplated by the invention, include one or more polynucleotides-of-interest that encode one or more polypeptides. In particular aspects, to achieve efficient translation of each of the plurality of polypeptides, the polynucleotide sequences can be separated by one or more IRES sequences or polynucleotide sequences encoding self-cleaving polypeptides.

As used herein, the term “Kozak sequence” refers to a short nucleotide sequence that greatly facilitates the initial binding of mRNA to the small subunit of the ribosome and increases translation. The consensus Kozak sequence is (GCC)RCCATGG (SEQ ID NO: 1), where R is a purine (A or G) (Kozak, 1986. Cell. 44(2):283-92, and Kozak, 1987. Nucleic Acids Res. 15(20):8125-48). In particular aspects, the vectors contemplated by the invention, comprise polynucleotides that have a consensus Kozak sequence and that encode a desired polypeptide.

In certain aspects, vectors comprise a selection gene, also termed a selectable marker. Typical selection genes encode proteins that (a) confer resistance to antibiotics or other toxins, e.g., ampicillin, neomycin, hygromycin, methotrexate, Zeocin, Blastocidin, or tetracycline, (b) complement auxotrophic deficiencies, or (c) supply critical nutrients not available from complex media, e.g., the gene encoding D-alanine racemase for Bacilli. Any number of selection systems may be used to recover transformed cell lines. These include, but are not limited to, the herpes simplex virus thymidine kinase (Wigler et al., 1977. Cell 11:223-232) and adenine phosphoribosyltransferase (Lowy et al., 1990. Cell 22:817-823) genes which can be employed in tk- or aprt-cells, respectively.

In various aspects, vectors of the invention are used to increase, establish and/or maintain the expression of one or more polypeptides. The terms “polypeptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers.

Particular aspects of the invention also include polypeptide “variants.” The recitation polypeptide “variant” refers to polypeptides that are distinguished from a reference polypeptide by the addition, deletion, truncations, and/or substitution of at least one amino acid residue, and that retain a biological activity. In certain aspects, a polypeptide variant is distinguished from a reference polypeptide by one or more substitutions, which may be conservative or non-conservative, as known in the art.

In certain aspects, a variant polypeptide includes an amino acid sequence having at least about 50%, 55%, 60%, 65%, 70%, 75%, 80%, 85%, 90%, 91%, 92%, 93%, 94%, 95%, 96%, 97%, 98%, 99% or more sequence identity or similarity to a corresponding sequence of a reference polypeptide. In certain aspects, amino acid additions or deletions occur at the C-terminal end and/or the N-terminal end of the reference polypeptide.

As noted above, polypeptides of the invention may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

A “host cell” includes cells transfected, infected, or transduced in vivo, ex vivo, or in vitro with a recombinant vector or a polynucleotide of the invention. Host cells may include packaging cells, producer cells, and cells infected with viral vectors. In particular aspects, host cells infected with viral vector of the invention are administered to a subject in need of therapy. In certain aspects, the term “target cell” is used interchangeably with host cell and refers to transfected, infected, or transduced cells of a desired cell type.

Large scale viral particle production is often necessary to achieve a reasonable viral titer. Viral particles are produced by transfecting a transfer vector into a packaging cell line that comprises viral structural and/or accessory genes, e.g., gag, pol, env, tat, rev, vif, vpr, vpu, vpx, or nef genes or other retroviral genes.

As used herein, the term “packaging vector” refers to an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. Methods for transfection, transduction or infection are well known by those of skill in the art. A retroviral/lentiviral transfer vector of the present invention can be introduced into a packaging cell line, via transfection, transduction or infection, to generate a producer cell or cell line. The packaging vectors of the present invention can be introduced into human cells or cell lines by standard methods including, e.g., calcium phosphate transfection, lipofection or electroporation. In some aspects, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self cleaving viral peptides.

Viral envelope proteins (env) determine the range of host cells which can ultimately be infected and transformed by recombinant retroviruses generated from the cell lines. In the case of lentiviruses, such as HIV-1, HIV-2, SIV, FIV and EIV, the env proteins include gp41 and gp120. Preferably, the viral env proteins expressed by packaging cells of the invention are encoded on a separate vector from the viral gag and pol genes, as has been previously described.

As used herein, the term “packaging cell lines” is used in reference to cell lines that do not contain a packaging signal, but do stably or transiently express viral structural proteins and replication enzymes (e.g., gag, pol and env) which are necessary for the correct packaging of viral particles. Any suitable cell line can be employed to prepare packaging cells of the invention. Generally, the cells are mammalian cells. In a particular aspect, the cells used to produce the packaging cell line are human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRCS cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In preferred aspects, the packaging cells are 293 cells, 293T cells, or A549 cells. In another preferred aspect, the cells are A549 cells.

As used herein, the term “producer cell line” refers to a cell line which is capable of producing recombinant retroviral particles, comprising a packaging cell line and a transfer vector construct comprising a packaging signal. The production of infectious viral particles and viral stock solutions may be carried out using conventional techniques. Methods of preparing viral stock solutions are known in the art and are illustrated by, e.g., Y. Soneoka et al. (1995) Nucl. Acids Res. 23:628-633, and N. R. Landau et al. (1992) J. Virol. 66:5110-5113. Infectious virus particles may be collected from the packaging cells using conventional techniques. For example, the infectious particles can be collected by cell lysis, or collection of the supernatant of the cell culture, as is known in the art. Optionally, the collected virus particles may be purified if desired. Suitable purification techniques are well known to those skilled in the art.

By “enhance” or “promote,” or “increase” or “expand” refers generally to the ability of the compositions and/or methods of the invention to elicit, cause, or produce higher numbers of transduced cells compared to the number of cells transduced by either vehicle or a control molecule/composition. In one embodiment, a hematopoietic stem cell transduced with compositions and methods of the present invention comprises an increase in the number of transduced cells compared to existing transduction compositions and methods. Increases in cell transduction, can be ascertained using methods known in the art, such as reporter assays, RT-PCR, and cell surface protein expression, among others. An “increased” or “enhanced” amount of transduction is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the number of cells transduced by vehicle, a control composition, or other transduction method.

By “decrease” or “lower,” or “lessen,” or “reduce,” or “abate” refers generally to compositions or methods that result in comparably fewer transduced cells compared to cells transduced with compositions and/or methods according to the present invention. A “decrease” or “reduced” amount of transduced cells is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the number of transduced cells (reference response) produced by compositions and/or methods according to the present invention.

By “maintain,” or “preserve,” or “maintenance,” or “no change,” or “no substantial change,” or “no substantial decrease” refers generally to a physiological response that is comparable to a response caused by either vehicle, a control molecule/composition, or the response in a particular cell lineage. A comparable response is one that is not significantly different or measurable different from the reference response.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Preferably, the subject is a human. A “subject in need thereof” is a subject suffering from or at risk of developing or suffering from an ocular disease or disorder. A subject at risk of developing or suffering from an ocular disease or disorder can be diagnosed by a physician or ocular specialist using routine methods in the art.

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, the term “amount” refers to “an amount effective” or “an effective amount” of a virus or transduced therapeutic cell to achieve a beneficial or desired prophylactic or therapeutic result, including clinical results.

A “prophylactically effective amount” refers to an amount of a virus or transduced therapeutic cell effective to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a virus or transduced therapeutic cell may vary according to factors such as the disease state, age, sex, and weight of the individual, and the ability of the stem and progenitor cells to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject (e.g., a patient).

The articles “a,” “an,” and “the” are used herein to refer to one or to more than one (i.e. to at least one) of the grammatical object of the article. By way of example, “an element” means one element or more than one element.

The use of the alternative (e.g., “or”) should be understood to mean either one, both, or any combination thereof of the alternatives. As used herein, the terms “include” and “comprise” are used synonymously.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one aspect, the term “about” or “approximately” refers a range of quantity, level, value, number, frequency, percentage, dimension, size, amount, weight or length .+−0.15%, .+−0.10%, .+−0.9%, .+−0.8%, .+−0.7%, .+−0.6%, .+−0.5%, .+−0.4%, .+−0.3%, .+−0.2%, or .+−0.1% about a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements

### EXAMPLES

### Example 1: Evaluation of Proteasome Inhibitors on AAV-Mediated Transduction Efficiency in Retinal Bipolar Cells

The expression of the transgene, mCherry, was used to evaluate the MV transduction efficiency. Targeted expression of mCherry in retinal bipolar cells was achieved by rMV2 vectors carrying an mGiuR6 promoter. rMV vectors at the concentration of 5×10¹² vg (viral-Genome contacting particle)/ml with or without containing proteasome inhibitors were intravitreally injected into the eyes of C57BL/6J mice at about one month of age. Animals were euthanized about one month after virus injection for assessing the expression of mCherry.

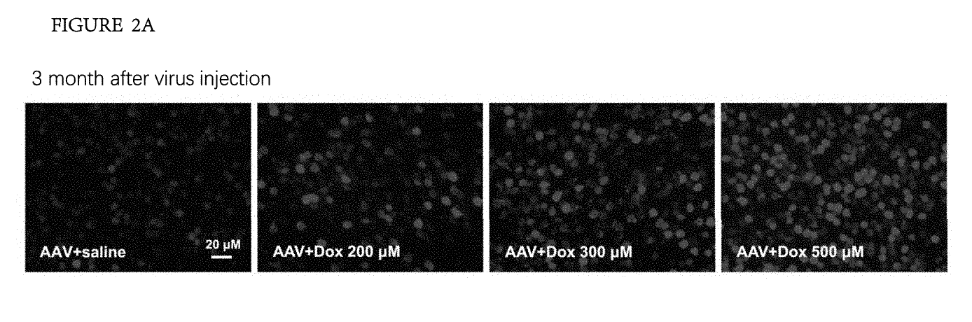

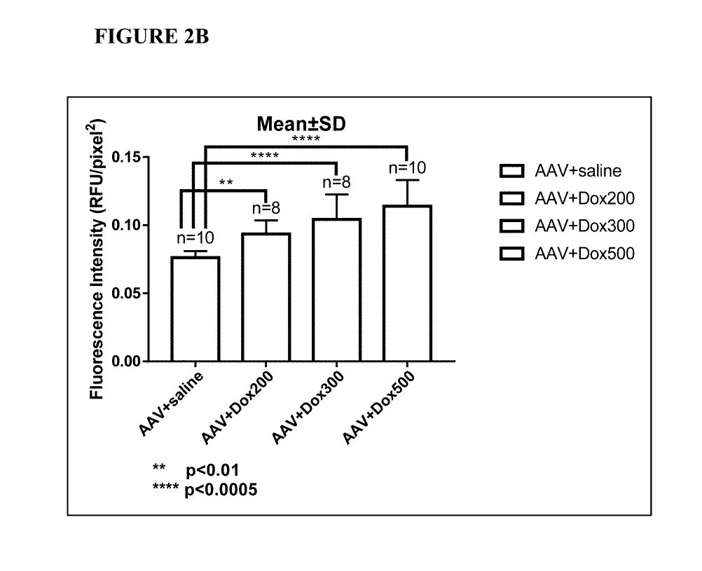

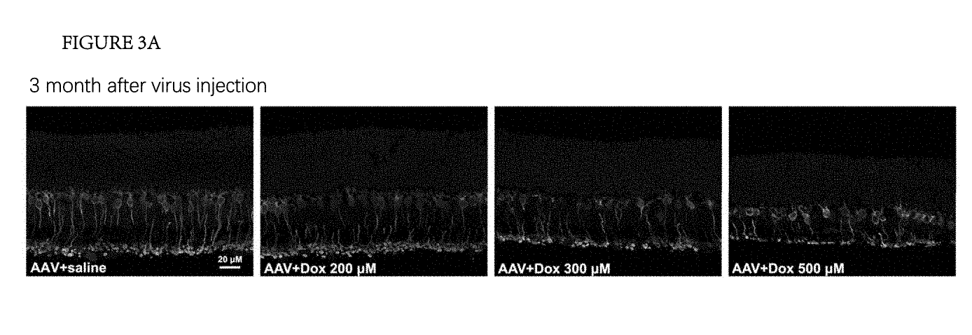

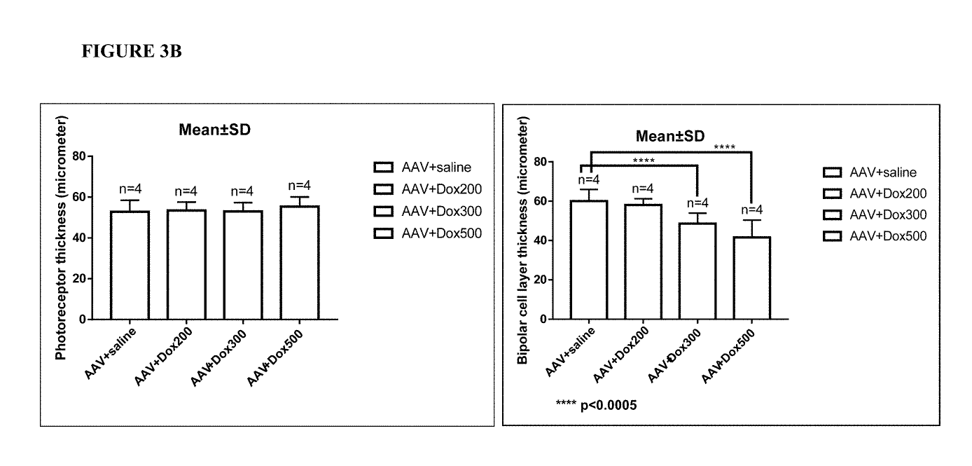

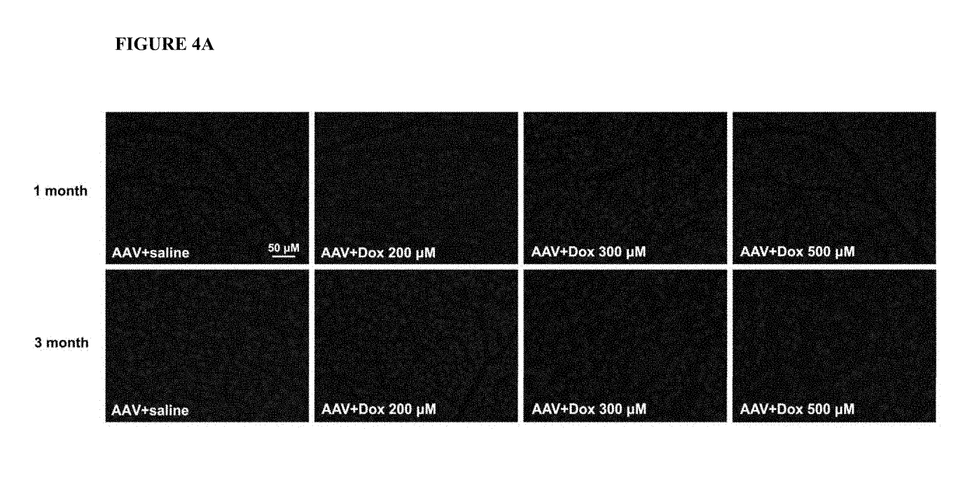

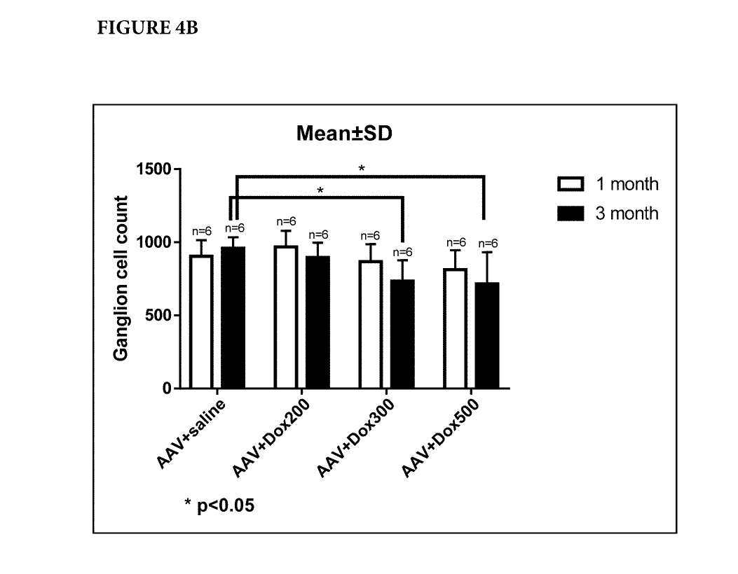

Results: We tested the effects of three proteasome inhibitors, MG132, doxorubicin, and aclarubicin, on rMV-mediated transduction efficiency in retinal bipolar cells. Retinas treated with doxorubicin from 200 μM to 800 μM exhibited a concentration-dependent increase in the transduction efficiency. Doxorubicin at the concentration of 2000M produced cytotoxicity as evidenced by the thinning of the retinas and decreased the number of mCherry-expressing bipolar cells. The optimal concentration of doxorubicin to enhance the MV transduction efficiency was 500 μM. MG132 (100 μM, 200 μM, 500 μM) and aclarubicin (50 μM, 100 μM) were not found to enhance the transduction efficiency, (FIGS. 1-4).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of enhancing the delivery of a gene of interest to an eye of a subject comprising administering a proteasome inhibitor and a viral vector encoding the gene of interest to the eye.

2. The method of claim 1, wherein the proteasome inhibitor is doxorubicin (DOX), aclarubicin, bortezomib, lactacystin, disulfiram epigallocatechin-3-gallate marizomib (salinosporamide A), oprozomib (ONX-0912), delanzomib (CEP-18770) epoxomicin, MG132, beta-hydroxy beta-methylbutyrate or carfilzomib.

3. The method of claim 1, wherein the gene of interest is an opsin.

4. The method of claim 3, wherein the opsin is selected from the group consisting of channelrhodopsin, halorhodopsin, melanopsin, pineal opsin, bacteriorhodopsin, and proteorhodopsin, or a functional variant thereof.

5. The method of claim 1, wherein the gene of interest is operably linked to a cell-specific promoter.

6. The method of claim 1, wherein the viral vector is encapsulated in a nanoparticle, a polymer, or a liposome.

7. The method of claim 1, wherein the subject is suffering from an ocular disease or disorder.

8. The method of claim 7, wherein the ocular disease is retinoblastoma, ocular melanoma, diabetic retinopathy, hypertensive retinopathy, or an inflammation of ocular tissue.

9. The method of claim 1, wherein the proteasome inhibitor and the viral vector are delivered concurrently or sequentially.

10. The method of claim 1, wherein the viral vector is delivered to a retinal cell.

11. The method of claim 10, wherein the retinal cell is a retinal ganglion cell, a retinal bipolar cell, a retinal horizontal cell, an amacrine cell, a photoreceptor cell, a Müller glial cell, or a retinal pigment epithelial cell.

12. The method of claim 1 or 9, wherein the proteasome inhibitor and the viral vector are administered to the vitreous of the eye.

13. The method of claim 1 or 9, wherein the proteasome inhibitor and the viral vector are administered by a route wherein the administration is by injection or infusion.

14. The method of claim 1 or 9, wherein the proteasome inhibitor and the viral vector are not administered subretinally.

15. A method of increasing light sensitivity or improving or restoring vision in a subject comprising administering a proteasome inhibitor and a viral vector that encodes an opsin to the vitreous of the eye.

16. The method of claim 15, wherein said opsin is selected from the group consisting of channelrhodopsin, halorhodopsin, melanopsin, pineal opsin, bacteriorhodopsin, and proteorhodopsin, or a functional variant thereof.

17. The method of claim 15, wherein the subject has an ocular disease or disorder.

18. The method of any one of claims 15-17, wherein the ocular disease is retinoblastoma, ocular melanoma, diabetic retinopathy, hypertensive retinopathy, or an inflammation of ocular tissues.


SEQUENCE LISTING (15 sequences)

SEQ ID NO: 1 - DNA, 10 bp, Artificial Sequence
  Synthetic Polynucleotide, where R is a purine (A or G)
gccrccatgg

SEQ ID NO: 2 - DNA, 1784 bp, Homo sapiens
caagcaggaggctgctgtgtgctgggagctgtcaggctcgtcctgaacagggaagggcccatccacctcccaaacccagtttatgcagtccttcgcaatgtcaggctcagggcctggcaccagccaagctccccacccttcccactgttaaaatggataggagcagggctaggcccagcctgttgactctgggcttccaccaggagaagtggttctggcagtagaaactatcggggcctgggagaggcgggggaagagagaaaggtggcatgtttcttgcttgctccctctaccagccttgtccaaatccccgcagccaccctaatccagcctgtctaatggagcccaagccggctcaggccctcggacgaggagcctgctaatccctgtggctaggagctcaccacctgtctccaggacgccctttgctctcttggcatcagagagccaaatcctgggcctcggatggggggatgataaaagcatcttttggccaagccccctcaccttggcctccacgatgagatggggagttaggtgcagagagcgttggcacagtgagcaccgcagctcgagtggctgcctcagacccagagcccgaggagactttatacggagccagaacgaccccgcggggttccatcctcccaagcaataggcgggagtgggagctgcgaggaaagccggcccctcccctccctccatccaaggcagtgtgggctgtttgtttcatgccattctgggtgtgaatcctgatgcccacacatgccagctgcatgcacttgggcaactcaactcactcctcgagggctgtttctcgactgcagggtgttgtaagttcgctaatactaaaggcttctccctcctggccccttcctgcccctcgctcttcctcctcttccttaggccctcccagctcaggcagcccctgccccctgcagggttctgcaaggagaaagctggggaataccttaggcaactgcagtcaggagcactggtggccaggacagagacagagagacagaaaaggggtcagggacagagagagataaccgcagggagagacaggaagggacagagacagaaaagatttccaagaagaggacagaggcagaaagccagggacagagactgagaaacagagacctagaggcagaagaagactgagatagagatggacagagattgtgtcagacacagccccagagacagccagacagtctgagtcagacgcaaaccaaagacaagaaaacaggaaaacagacccagagattgggagagggaggggaaggagatgcggggagagccagcaccgccaccccccacactcaggaggggtctccaccctcggagcggtctctcatccctccctagaatccttaaatcctctctcgctcagggcctcggccgcatctgtcacagacttgtcctgaaccgacagcggctggcgcaggtgactggcttggggcgggagcctgggtgtgcgctggggatggaccccgaggaagaggggccaagctgtcgggaagcggcagggctggaggggtggaggcagtggtcgggcgggaccccgggcgacagggttcggcgcttgtaagagcgagacggaggcccgggcaggccggctgagctaactccccagagccgaagtggaaggcgcgccccgagcgccttctccccaggaccccggtgtccctccccgcgccccgagcccgcgctctccttcccccgccctcagagcgctccccgcccctctgtctccccgcagcccgctagacgagccg

SEQ ID NO: 3 - DNA, 3773 bp, Chlamydomonas reinhardtii
cttgactacgcttcgctgtaataatagcagcgccacaagtagtgtcgccagacaactctcactttgagcttgagcacaccgctgagccccgatgtcgcggaggccatggcttcttgccctagcgctggcagtggcgctggcggccggcagcgcaggagcctcgactggcagtgacgcgacggtgccggtcgcgactcaggatggccccgactacgttttccaccgtgcccacgagcgcatgctcttccaaacctcatacactcttgagaacaatggttctgttatttgcatcccgaacaacggccagtgcttctgcttggcttggcttaaatccaacggaacaaatgccgagaagttggctgccaacattctgcagtggattacttttgcgctttcagcgctctgcctgatgttctacggctaccagacctggaagtctacttgcggctgggaggagatttacgtggccacgatcgagatgatcaagttcatcatcgagtatttccatgagtttgacgaacctgcggtgatctactcatccaacggcaacaagaccgtgtggcttcgttacgcggagtggctgctgacctgccctgtcattcttatccatctgagcaaccttacgggtctggcgaacgactataacaagcgtaccatgggtctgctggtgtcagatatcggcacgatcgtgtggggcaccacggccgcgctgtccaagggatacgtccgtgtcattttcttcctgatgggcctgtgctacggcatctacacattcttcaacgcagccaaggtctacattgaggcgtaccacaccgtgcccaagggcatttgccgcgacctggtccgctaccttgcctggctctacttctgttcatgggctatgttcccggtgctgttcctgctgggccccgagggctttggccacatcaaccaattcaactctgccatcgcccacgccatcctggaccttgcctccaagaacgcttggagtatgatgggtcactttctgcgtgtcaagatccacgagcacatcctgctgtacggcgacatccgcaagaagcagaaggtcaacgtggctggccaggagatggaggtggagaccatggtgcacgaggaggacgacgagacgcagaaggtgcccacggcaaagtacgccaaccgcgactcgttcatcatcatgcgcgaccgcctcaaggagaagggcttcgagacccgcgcctcgctggacggcgacccgaacggcgacgccgaggccaacgctgcagccggcggcaagcccggaatggagatgggcaagatgaccggcatgggcatgggcatgggtgccggcatgggcatggcgaccatcgattcgggccgcgtcatcctcgccgtgccggacatctccatggtggactttttccgcgagcagttcgcgcggctgcccgtgccctacgaactggtgcccgcgctgggcgcggagaacaccctccagctggtgcagcaggcgcagtcactgggaggctgcgacttcgtcctcatgcaccccgagttcctgcgcgaccgcagtcccacgggtctgctgccccgcctcaagatgggcgggcagcgcgccgcggccttcggctgggcggcaatcggccccatgcgggacttgatcgagggttcgggcgttgacggctggctggagggccccagctttggcgccggcatcaaccagcaggcgctggtggcgctgatcaaccgcatgcagcaggccaagaagatgggcatgatgggcggtatgggtatgggcatgggcggcggcatgggtatgggcatgggtatgggcatgggcatggcccccagcatgaacgccggcatgactggcggcatgggcggcgcctccatgggcggtgccgtgatgggcatgggcatgggcatgcagcccatgcagcaggctatgccggccatgtcgcccatgatgactcagcagcccagcatgatgagtcagccctccgccatgagcgccggcggcgccatgcaggccatgggtggcgtcatgcccagccccgcccccggcggccgcgtgggcaccaacccgctgtttggctctgcgccctctccgctgagctcgcagcccggcatcagccctggcatggcgacgccgcccgccgccaccgccgcacccgccgctggcggcagcgaggccgagatgctgcagcagctgatgagcgagatcaaccgcctgaagaacgagctgggcgagtaaactgctggcccagccgtacggacatatgcctgctgaggcaccagcgccgcaacacacatcgccgcagctgtcgcggctgccatgttggatttgcgcgtggcggcgtggtggtgtggtggtgtggtggcaggaacaagggcgaagctttaacttacccggcgctcagcgcttcgttcataggttcggcgcttgagccgtggtagcggcaagtgtgccgcggcaacgcggggcaaagcgaagacgccgatgacttgacgcctggtatgacaccttggtctatgaagtcgcgctgcggtgctgggatcaagaaacagcaactcgaggaaggtatcatcgagcgtcgttatacagcagacaaggtacgaaacggtgtgcaggagggcatgcacagcagcttcaaatggcacgtgcatggctctgttgcgaacaagctgctctgagacacggattgagagcccttaatcggtggtcacaagaggtggggttacggtatcggggcgctgcgatagtcctgcaagtgctgcctgttgaacacaagggctcagaatttatggcagggaaggtcaaggccgagaatggccgcgtgcgtgatttattgtttgagccagggcttgttgatactgtattaatcatgcgtgtgtgtttgtgtgcgtgaacgtgacccgacggattccgtgagccgctgcgcatgcaagatccggccctgacctatgtcctagtacaagccgatcgtgcttggcctgccttgattaatgcgtcgcctgaggattcccgtttgtggcttttaaggagcgcgaatacggcagttacgtgacctgcttgtcgggttggggaaatccgtctggtgtgtacctggcctggccggctgatcgggtctgcttccggcaagtaactgtgcgggtgaaactacaaaaggcagcgccggttgtgggcgtcgttttggttggtttggcggggttcccattgcaatgtgtgtttccataaatcatgggcgacactggatggaacggctttggcttgcgcggaggcttctcaggtcggtacctaatattgccataacctctctttcaaacctgcgcctcctgcaatcaatagatgcagggggctgcgcatcaaccctggggaccatacaatgcttaattccgctctgcaattattcgagtagtggcctgtcgcggagaagctgcttcagggtgtcaatgtggctgcaggacggcacaataaaagagagtgtgggagcaccgtatcctgaacagcggtggattctcagagcctgtgggcgcttgcccggcgcaccggccgctcgtggggggtagcagctgcggctggtgtgctgatcttcatttgtttctgtttgggggggcaccccttgctctcgttggtgtgagcgccggtgcgcagttgtaataagggaagggagcataacgcggcgtggcttacactaagagagttgatactttgaatcgacgccttggatgcatgtaaaaccagaatttgaaaaaaaaaaaaaaaaaaaaaaaaaaaaaaaaaaaaaaaaaaaaaa

SEQ ID NO: 4 - DNA, 2236 bp, Chlamydomonas reinhardtii
gcgttgcttgactacgcttcgctgtaataatagcagcgccacaagtagtgtcgccaaacaactctcactttgagcttgagcacaccgctgagccccgatgtcgcggaggccatggcttcttgccctagcgctggcagtggcgctggcggccggcagcgcaggagcctcgactggcagtgacgcgacggtgccggtcgcgactcaggatggccccgactacgttttccaccgtgcccacgagcgcatgctcttccaaacctcatacactcttgagaacaatggttctgttatttgcatcccgaacaacggccagtgcttctgcttggcttggcttaaatccaacggaacaaatgccgagaagttggctgccaacattctgcagtggattacttttgcgctttcagcgctctgcctgatgttctacggctaccagacctggaagtctacttgcggctgggaggagatttacgtggccacgatcgagatgatcaagttcatcatcgagtatttccatgagtttgacgaacctgcggtgatctactcatccaacggcaacaagaccgtgtggcttcgttacgcggagtggctgctgacctgccctgtcattcttatccatctgagcaaccttacgggtctggcgaacgactataacaagcgtaccatgggtctgctggtgtcagatatcggcacgatcgtgtggggcaccacggccgcgctgtccaagggatacgtccgtgtcattttcttcctgatgggcctgtgctacggcatctacacattcttcaacgcagccaaggtctacattgaggcgtaccacaccgtgcccaagggcatttgccgcgacctggtccgctaccttgcctggctctacttctgttcatgggctatgttcccggtgctgttcctgctgggccccgagggctttggccacatcaaccaattcaactctgccatcgcccacgccatcctggaccttgcctccaagaacgcttggagtatgatgggtcactttctgcgtgtcaagatccacgagcacatcctgctgtacggcgacatccgcaagaagcagaaggtcaacgtggctggccaggagatggaggtggagaccatggtgcacgaggaggacgacgagacgcagaaggtgcccacggcaaagtacgccaaccgcgactcgttcatcatcatgcgcgaccgcctcaaggagaagggcttcgagacccgcgcctcgctggacggcgacccgaacggcgacgccgaggccaacgctgcagccggcggcaagcccggaatggagatgggcaagatgaccggcatgggcatgggcatgggtgccggcatgggcatggcgaccatcgattcgggccgcgtcatcctcgccgtgccggacatctccatggtggactttttccgcgagcagttcgcgcggctgcccgtgccctacgaactggtgcccgcgctgggcgcggagaacaccctccagctggtgcagcaggcgcagtcactgggaggctgcgacttcgtcctcatgcaccccgagttcctgcgcgaccgcagtcccacgggtctgctgccccgcctcaagatgggcgggcagcgcgccgcggccttcggctgggcggcaatcggccccatgcgggacttgatcgagggttcgggcgttgacggctggctggagggccccagctttggcgccggcatcaaccagcaggcgctggtggcgctgatcaaccgcatgcagcaggccaagaagatgggcatgatgggcggtatgggtatgggcatgggcggcggcatgggtatgggcatgggtatgggcatgggcatggcccccagcatgaacgccggcatgactggcggcatgggcggcgcctccatgggcggtgccgtgatgggcatgggcatgggcatgcagcccatgcagcaggctatgccggccatgtcgcccatgatgactcagcagcccagcatgatgagtcagccctccgccatgagcgccggcggcgccatgcaggccatgggtggcgtcatgcccagccccgcccccggcggccgcgtgggcaccaacccgctgtttggctctgcgccctctccgctgagctcgcagcccggcatcagccctggcatggcgacgccgcccgccgccaccgccgcacccgccgctggcggcagcgaggccgagatgctgcagcagctgatgagcgagatcaaccgcctgaagaacgagctgggcgagtaa

SEQ ID NO: 5 - DNA, 2448 bp, Chlamydomonas reinhardtii
catctgtcgccaagcaagcattaaacatggattatggaggcgccctgagtgccgttgggcgcgagctgctatttgtaacgaacccagtagtcgtcaatggctctgtacttgtgcctgaggaccagtgttactgcgcgggctggattgagtcgcgtggcacaaacggtgcccaaacggcgtcgaacgtgctgcaatggcttgctgctggcttctccatcctactgcttatgttttacgcctaccaaacatggaagtcaacctgcggctgggaggagatctatgtgtgcgctatcgagatggtcaaggtgattctcgagttcttcttcgagtttaagaacccgtccatgctgtatctagccacaggccaccgcgtccagtggttgcgttacgccgagtggcttctcacctgcccggtcattctcattcacctgtcaaacctgacgggcttgtccaacgactacagcaggcgcaccatgggtctgcttgtgtctgatattggcacaattgtgtggggcgccacttccgccatggccaccggatacgtcaaggtcatcttcttctgcctgggtctgtgttatggtgctaacacgttctttcacgctgccaaggcctacatcgagggttaccacaccgtgccgaagggccggtgtcgccaggtggtgactggcatggcttggctcttcttcgtatcatggggtatgttccccatcctgttcatcctcggccccgagggcttcggcgtcctgagcgtgtacggctccaccgtcggccacaccatcattgacctgatgtcgaagaactgctggggtctgctcggccactacctgcgcgtgctgatccacgagcatatcctcatccacggcgacattcgcaagaccaccaaattgaacattggtggcactgagattgaggtcgagacgctggtggaggacgaggccgaggctggcgcggtcaacaagggcaccggcaagtacgcctcccgcgagtccttcctggtcatgcgcgacaagatgaaggagaagggcattgacgtgcgcgcctctctggacaacagcaaggaggtggagcaggagcaggccgccagggctgccatgatgatgatgaacggcaatggcatgggtatgggaatgggaatgaacggcatgaacggaatgggcggtatgaacgggatggctggcggcgccaagcccggcctggagctcactccgcagctacagcccggccgcgtcatcctggcggtgccggacatcagcatggttgacttcttccgcgagcagtttgctcagctatcggtgacgtacgagctggtgccggccctgggcgctgacaacacactggcgctggttacgcaggcgcagaacctgggcggcgtggactttgtgttgattcaccccgagttcctgcgcgaccgctctagcaccagcatcctgagccgcctgcgcggcgcgggccagcgtgtggctgcgttcggctgggcgcagctggggcccatgcgtgacctgatcgagtccgcaaacctggacggctggctggagggcccctcgttcggacagggcatcctgccggcccacatcgttgccctggtggccaagatgcagcagatgcgcaagatgcagcagatgcagcagattggcatgatgaccggcggcatgaacggcatgggcggcggtatgggcggcggcatgaacggcatgggcggcggcaacggcatgaacaacatgggcaacggcatgggcggcggcatgggcaacggcatgggcggcaatggcatgaacggaatgggtggcggcaacggcatgaacaacatgggcggcaacggaatggccggcaacggaatgggcggcggcatgggcggcaacggtatgggtggctccatgaacggcatgagctccggcgtggtggccaacgtgacgccctccgccgccggcggcatgggcggcatgatgaacggcggcatggctgcgccccagtcgcccggcatgaacggcggccgcctgggtaccaacccgctcttcaacgccgcgccctcaccgctcagctcgcagctcggtgccgaggcaggcatgggcagcatgggaggcatgggcggaatgagcggaatgggaggcatgggtggaatggggggcatgggcggcgccggcgccgccacgacgcaggctgcgggcggcaacgcggaggcggagatgctgcagaatctcatgaacgagatcaatcgcctgaagcgcgagcttggcgagtaaaaggctggaggccggtactgcgatacctgcgagctcgcgcgcctgactcgtcgtacacacggctcaggagcacgcgcgcgtggacttctcaacctgtgtgcaacgtatctagagcggcctgtgcgcgaccgtccgtgagcattccggtgcgatcttcccgccttcgcaccgcaagttcccttcctggccctgctgcgcctgacgcatc

SEQ ID NO: 6 - DNA, 2241 bp, Chlamydomonas reinhardtii
gcatctgtcgccaagcaagcattaaacatggattatggaggcgccctgagtgccgttgggcgcgagctgctatttgtaacgaacccagtagtcgtcaatggctctgtacttgtgcctgaggaccagtgttactgcgcgggctggattgagtcgcgtggcacaaacggtgcccaaacggcgtcgaacgtgctgcaatggcttgctgctggcttctccatcctactgcttatgttttacgcctaccaaacatggaagtcaacctgcggctgggaggagatctatgtgtgcgctatcgagatggtcaaggtgattctcgagttcttcttcgagtttaagaacccgtccatgctgtatctagccacaggccaccgcgtccagtggttgcgttacgccgagtggcttctcacctgcccggtcattctcattcacctgtcaaacctgacgggcttgtccaacgactacagcaggcgcaccatgggtctgcttgtgtctgatattggcacaattgtgtggggcgccacttccgccatggccaccggatacgtcaaggtcatcttcttctgcctgggtctgtgttatggtgctaacacgttctttcacgctgccaaggcctacatcgagggttaccacaccgtgccgaagggccggtgtcgccaggtggtgactggcatggcttggctcttcttcgtatcatggggtatgttccccatcctgttcatcctcggccccgagggcttcggcgtcctgagcgtgtacggctccaccgtcggccacaccatcattgacctgatgtcgaagaactgctggggtctgctcggccactacctgcgcgtgctgatccacgagcatatcctcatccacggcgacattcgcaagaccaccaaattgaacattggtggcactgagattgaggtcgagacgctggtggaggacgaggccgaggctggcgcggtcaacaagggcaccggcaagtacgcctcccgcgagtccttcctggtcatgcgcgacaagatgaaggagaagggcattgacgtgcgcgcctctctggacaacagcaaggaggtggagcaggagcaggccgccagggctgccatgatgatgatgaacggcaatggcatgggtatgggaatgggaatgaacggcatgaacggaatgggcggtatgaacgggatggctggcggcgccaagcccggcctggagctcactccgcagctacagcccggccgcgtcatcctggcggtgccggacatcagcatggttgacttcttccgcgagcagtttgctcagctatcggtgacgtacgagctggtgccggccctgggcgctgacaacacactggcgctggttacgcaggcgcagaacctgggcggcgtggactttgtgttgattcaccccgagttcctgcgcgaccgctctagcaccagcatcctgagccgcctgcgcggcgcgggccagcgtgtggctgcgttcggctgggcgcagctggggcccatgcgtgacctgatcgagtccgcaaacctggacggctggctggagggcccctcgttcggacagggcatcctgccggcccacatcgttgccctggtggccaagatgcagcagatgcgcaagatgcagcagatgcagcagattggcatgatgaccggcggcatgaacggcatgggcggcggtatgggcggcggcatgaacggcatgggcggcggcaacggcatgaacaacatgggcaacggcatgggcggcggcatgggcaacggcatgggcggcaatggcatgaacggaatgggtggcggcaacggcatgaacaacatgggcggcaacggaatggccggcaacggaatgggcggcggcatgggcggcaacggtatgggtggctccatgaacggcatgagctccggcgtggtggccaacgtgacgccctccgccgccggcggcatgggcggcatgatgaacggcggcatggctgcgccccagtcgcccggcatgaacggcggccgcctgggtaccaacccgctcttcaacgccgcgccctcaccgctcagctcgcagctcggtgccgaggcaggcatgggcagcatgggaggcatgggcggaatgagcggaatgggaggcatgggtggaatggggggcatgggcggcgccggcgccgccacgacgcaggctgcgggcggcaacgcggaggcggagatgctgcagaatctcatgaacgagatcaatcgcctgaagcgcgagcttggcgagtaa

SEQ ID NO: 7 - protein, 315 aa, Artificial Sequence
  Synthetic fragment of Chop2 protein
MDYGGALSAVGRELLFVTNPVVVNGSVLVREDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVLVIFFCLGLCYGANTFFHAALAYIEGYHTVPLGRCAQVVTGMAWLFFVSWGMFPILFILGPEGFGVLSVYGSTVGHTIIDLMSLNCWGLLGHYLRVLIHEHILIHGDIRKTTKLNIGGTEIEVETLVEDEAEAGAVNKGTGK

SEQ ID NO: 8 - protein, 712 aa, Chlamydomonas reinhardtii
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVILIHLSNLTGLANDYNKRTMGLLVSDIGTIVWGTTAALSKGYVRVIFFLMGLCYGIYTFFNAAKVYIEAYHTVPKGICRDLVRYLAWLYFCSWAMFPVLFLLGPEGFGHINQFNSAIAHAILDLASKNAWSMMGHFLRVKIHEHILLYGDIRKKQKVNVAGQEMEVETMVHEEDDETQKVPTAKYANRDSFIIMRDRLKEKGFETRASLDGDPNGDAEANAAAGGKPGMEMGKMTGMGMGMGAGMGMATIDSGRVILAVPDISMVDFFREQFARLPVPYELVPALGAENTLQLVQQAQSLGGCDFVLMHPEFLRDRSPTGLLPRLKMGGQRAAAFGWAAIGPMRDLIEGSGVDGWLEGPSFGAGINQQALVALINRMQQAKKMGMMGGMGMGMGGGMGMGMGMGMGMAPSMNAGMTGGMGGASMGGAVMGMGMGMQPMQQAMPAMSPMMTQQPSMMSQPSAMSAGGAMQAMGGVMPSPAPGGRVGTNPLFGSAPSPLSSQPGISPGMATPPAATAAPAAGGSEAEMLQQLMSEINRLKNELGE

SEQ ID NO: 9 - protein, 712 aa, Chlamydomonas reinhardtii; identical to SEQ ID NO: 8

SEQ ID NO: 10 - protein, 737 aa, Chlamydomonas reinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGKYASRESFLVMRDKMKEKGIDVRASLDNSKEVEQEQAARAAMMMMNGNGMGMGMGMNGMNGMGGMNGMAGGAKPGLELTPQLQPGRVILAVPDISMVDFFREQFAQLSVTYELVPALGADNTLALVTQAQNLGGVDFVLIHPEFLRDRSSTSILSRLRGAGQRVAAFGWAQLGPMRDLIESANLDGWLEGPSFGQGILPAHIVALVAKMQQMRKMQQMQQIGMMTGGMNGMGGGMGGGMNGMGGGNGMNNMGNGMGGGMGNGMGGNGMNGMGGGNGMNNMGGNGMAGNGMGGGMGGNGMGGSMNGMSSGVVANVTPSAAGGMGGMMNGGMAAPQSPGMNGGRLGTNPLFNAAPSPLSSQLGAEAGMGSMGGMGGMSGMGGMGGMGGMGGAGAATTQAAGGNAEAEMLQNLMNEINRLKRELGE

SEQ ID NO: 11 - protein, 737 aa, Chlamydomonas reinhardtii; identical to SEQ ID NO: 10

SEQ ID NO: 12 - DNA, 876 bp, Artificial Sequence
  Synthetic Polynucleotide
atgacagagaccctgcctcccgtgaccgagagtgccgtggcccttcaagccgaggttacccaaagggagttgttcgagttcgtgctgaacgaccctttgcttgcaagcagtctctatatcaacatcgcacttgcaggactgagtatactgctgttcgtttttatgacccgaggactcgatgatccacgggcaaaacttattgctgtgtcaaccatccttgtgcctgtcgtcagcattgcctcctacactggattggcgagcggcctgacaatttccgttcttgaaatgccagcgggccattttgcagaaggcagctcagtgatgctgggaggagaagaggtagatggtgtagtcaccatgtggggacggtatctcacctgggcactttccacgcccatgattctcctcgctctgggtctcctggccggaagcaatgctacaaagctcttcacagctatcactttcgatatcgctatgtgcgtgactggccttgccgcggccctgactacctcctcccacctcatgagatggttctggtacgctatcagttgtgcatgctttctggtggtcttgtatatcctgctggtggagtgggcacaggacgccaaagccgcgggaaccgctgacatgttcaataccctgaagctgttgacagtagtgatgtggctggggtatccaattgtgtgggctcttggagtcgagggtatcgcggtgttgcccgttggggtgacgagctggggatattctttcctggatatcgtggcaaagtacattttcgcattcttgctcctgaactatctgacgtcaaacgaatctgtcgtgtccggcagcattttggatgttccatctgcttctgggaccccggctgatgattaa

SEQ ID NO: 13 - protein, 291 aa, Artificial Sequence
  Synthetic Polypeptide
MTETLPPVTESAVALQAEVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADD

SEQ ID NO: 14 - DNA, 2137 bp, Artificial Sequence
  Synthetic Polynucleotide
cactcattcctttgcgcttcattggacattaagcagtcagcagcccaaagagcagctccaggctggatggatgagagcgggcagcaggtggaccaggccgcagggttaaggatggtatagagccggaagtctggggaccgatccctgatctttccatggccttagctcctctgagagcctgagcatggactctccttcaggaccaagagtcttgtcaagcttaactcaggatcccagcttcacaaccagtcctgccctgcaaggcatttggaacggcactcagaacgtctccgtaagagcccagcttctctctgttagccccacgacatctgcacatcaggctgctgcctgggtccccttccccacagtcgatgtcccagaccatgctcactataccctaggcacggtgatcctgctggtgggactcacagggatgctgggcaatctgacggtcatctacaccttctgcaggaacagaggcctgcggacaccagcaaacatgttcatcatcaacctcgcagtcagcgacttcctcatgtcagtcactcaggccccggtcttctttgccagcagcctctacaagaagtggctctttggggagacaggttgcgagttctatgccttctgcggggctgtctttggcatcacttccatgatcaccctgacagccatagccatggaccgctatctggtgatcacacgtccactggccaccatcggcaggggatccaaaagacgaacggcactcgtcctgctaggcgtctggctttatgccctggcctggagtctgccacctttctttggttggagtgcctacgtgcccgaggggctgctgacatcctgctcctgggactacatgaccttcacaccccaggtgcgtgcctacaccatgctgctcttctgctttgtcttcttcctccccctgctcatcatcatcttctgctacatcttcatcttcagggccatccgagagacaggccgggcctgtgagggctgcggtgagtcccctctgcggcagaggcggcagtggcagcggctgcagagtgagtggaagatggccaaggtcgcactgattgtcattcttctcttcgtgctgtcctgggctccctactccactgtggctctggtggcctttgctggatactcgcacatcctgacgccctacatgagctcggtgccagccgtcatcgccaaggcttctgccatccacaatcccattatctacgccatcactcaccccaagtacagggtggccattgcccagcacctgccttgccttggggtgcttctcggtgtatcaggccagcgcagccacccctccctcagctaccgctctacccaccgctccacattgagcagccagtcctcagacctcagctggatctctggacggaagcgtcaagagtccctgggttctgagagtgaagtgggctggacagacacagaaacaaccgctgcatggggagctgcccagcaagcaagtggacagtccttctgcagtcagaacctagaagatggagaactcaaggcctcttccagcccccaggtacagagatctaagactcccaaggtgcctggacccagtacctgccgccctatgaaaggacagggagccaggccaagtagcctaaggggtgaccagaaaggcaggcttgctgtgtgcacaggcctctcagagtgtccccatccccatacatcccagtttccccttgctttcctagaggatgatgtgactctcagacatctgtagcagggtctaagtatgatctgtatctaggggaatatctgcatgtgactgtgtagctctgcgcatgacatgctgtcagctatgttgtaccatatgtatatgtagagtatgcatataacttatgtgcccttgaagatatgtggcctacagcagagaacaactcatgcgtgtgtggaccatgttcctggcatatatgctctctgtcactgtgatgcctctgtgttgtgtgggtgacagagtgtgatggtgttcacctctctgcgcgggttttgatgctgggcaaacacggggaagggagctgcaagccatgtactagctcactgccgatggcctgtgctcaagatgtcaccgaggagaacacttgtagctattaaaagaaggccagctgtc

SEQ ID NO: 15 - protein, 521 aa, Artificial Sequence
  Synthetic Polypeptide
MDSPSGPRVLSSLTQDPSFTTSPALQGIWNGTQNVSVRAQLLSVSPTTSAHQAAAWVPFPTVDVPDHAHYTLGTVILLVGLTGMLGNLTVIYTFCRNRGLRTPANMFIINLAVSDFLMSVTQAPVFFASSLYKKWLFGETGCEFYAFCGAVFGITSMITLTAIAMDRYLVITRPLATIGRGSKRRTALVLLGVWLYALAWSLPPFFGWSAYVPEGLLTSCSWDYMTFTPQVRAYTMLLFCFVFFLPLLIIIFCYIFIFRAIRETGRACEGCGESPLRQRRQWQRLQSEWKMAKVALIVILLFVLSWAPYSTVALVAFAGYSHILTPYMSSVPAVIAKASAIHNPIIYAITHPKYRVAIAQHLPCLGVLLGVSGQRSHPSLSYRSTHRSTLSSQSSDLSWISGRKRQESLGSESEVGWTDTETTAAWGAAQQASGQSFCSQNLEDGELKASSSPQVQRSKTPKVPGPSTCRPMKGQGARPSSLRGDQKGRLAVCTGLSECPHPHTSQFPLAFLEDDVTLRHL